(19) United States
(12) Patent Application Publication
(10) Pub. No.: US 2004/0058876 A1
(43) Pub. Date: Mar. 25, 2004

(54) Secondary binding site of dipeptidyl peptidase IV (DP IV)

(76) Inventors: Torsten Hoffmann, Halle/Saale (DE); Kerstin Kuehn-Wache, Halle/Saale (DE); Hans-Ulrich Demuth, Halle/Saale (DE); Wolfgang Brandt, Halle/Saale (DE); Jochaim Baer, Halle/Saale (DE)

Correspondence Address: BROWN, RUDNICK, BERLACK & ISRAELS, LLP., BOX IP, 18TH FLOOR, ONE FINANCIAL CENTER, BOSTON, MA, 02111, US

(21) Appl. No.: 10/246,817

(22) Filed: Sep. 18, 2002

(51) Int. Cl. A61K 38/08; A61K 31/401; C07K 7/06

(52) U.S. Cl. 514/17; 514/423; 530/329; 548/530

(57) ABSTRACT

The present application relates to the secondary binding site of dipeptidyl peptidase IV, its relationship amongst substrates and to the modulation of substrate specificity of dipeptidyl peptidase IV (DP IV, synonym: DPP IV, CD26, EC 3.4.14.5). The application relates further to compounds that bind to the secondary binding site of DP IV and their use to modulate the substrate specificity of DP IV; methods of treatment of various DP IV mediated disorders; and screening methods for the identification of secondary binding sites on DP IV and DP IV-like enzymes.

### FIELD OF THE APPLICATION

The present application relates to the secondary binding site of dipeptidyl peptidase IV, its relationship with any type of substrates and to the modulation of substrate specificity of dipeptidyl peptidase IV (DP IV, synonym: DPP IV, CD26, EC 3.4.14.5).

The application relates further to compounds that bind to the secondary binding site of DP IV and their use to modulate the substrate specificity of DP IV.

Furthermore, the present invention provides a method for treating DP IV mediated disorders, selected from but not restricted to, impaired glucose tolerance, glucosuria, hyperlipidaemia, metabolic acidosis, diabetes mellitus, diabetic neuropathy and nephropathy and of sequelae caused by diabetes mellitus in mammals, metabolism-related hypertension and cardiovascular sequelae caused by hypertension in mammals, for the prophylaxis or treatment of skin diseases and diseases of the mucosae, autoimmune diseases and inflammatory conditions, and for the treatment of psychosomatic, neuropsychiatric and depressive illnesses, such as anxiety, depression, sleep disorders, chronic fatigue, schizophrenia, epilepsy, nutritional disorders, spasm and chronic pain, and a simple method for the treatment of those diseases in mammals.

The present application also provides a screening method for the identification of agents, which bind to the secondary binding site of dipeptidyl peptidase IV.

Further on, a screening method for the identification and determination of one or more secondary binding sites of DP IV-like enzymes is provided.

### BACKGROUND OF THE INVENTION

The exopeptidase dipeptidyl peptidase IV (DP IV, CD26, EC 3.4.14.5) is involved in a number of physiological regulation processes. On the one hand, DP IV is a peptidase which can change the activity of a number of peptide hormones, neuropeptides and chemokines in a very specific manner (Mentlein, Reg. Pep. 85, pp. 9-24 (1999) while on the other hand the DP IV protein molecule exerts protein-protein interactions, so mediating the regulation of intracellular signaling cascades. A growing number of peptide substrates containing proline, alanine or serine in the penultimate position are identified as substrates of DP IV in vitro and in vivo. Bioactive peptides which are substrates for DP IV and members of such regulation cascades are, among others, NPY, GIP, GLP-1, glucagons, VIP and PACAP. Furthermore, many DP IV-inhibitors belonging to different structural classes are known.

It is known that DP IV-Inhibitors may be useful for the treatment of impaired glucose tolerance and diabetes mellitus (International Patent Application, Publication Number WO 99/61431, Pederson R A et al, Diabetes. 1998 August; 47(8):1253-8 and Pauly R P et al, Metabolism 1999 March; 48(3):385-9). In particular WO 99/61431 discloses DP IV-Inhibitors comprising an amino acid residue and a thiazolidine or pyrrolidine group, and salts thereof, especially L-threo-isoleucyl thiazolidine, L-allo-isoleucyl thiazolidine, L-threo-isoleucyl pyrrolidine, L-allo-isoleucyl thiazolidine, L-allo-isoleucyl pyrrolidine, and salts thereof.

Further examples of low molecular weight dipeptidyl peptidase IV inhibitors are agents such as tetrahydroisoquinolin-3-carboxamide derivatives, N-substituted 2-cyanopyroles and—pyrrolidines, N-(N′-substituted glycyl)-2-cyanopyrrolidines, N-(substituted glycyl)-thiazolidines, N-(substituted glycyl)-4-cyanothiazolidines, amino-acyl-borono-prolyl-inhibitors, cyclopropyl-fused pyrrolidines and heterocyclic compounds. Inhibitors of dipeptidyl peptidase IV are described in U.S. Pat. No. 6,380,398; U.S. Pat. No. 6,011,155; U.S. Pat. No. 6,107,317; U.S. Pat. No. 6,110,949; U.S. Pat. No. 6,124,305; U.S. Pat. No. 6,172,081; WO 95/15309, WO 99/61431, WO 99/67278, WO 99/67279, DE 198 34 591, WO 97/40832, DE 196 16 486 C 2, WO 98/19998, WO 00/07617, WO 99/38501, WO 99/46272, WO 99/38501, WO 01/68603, WO 01/40180, WO 01/81337, WO 01/81304, WO 01/55105, WO 02/02560 and WO 02/14271, the teachings of which are herein incorporated by reference in their entirety, especially concerning these inhibitors, their definition, uses and their production.

Other Potential target diseases and the actual stage of research are summarized in Table 1.

TABLE 1
Target diseases for DP IV-inhibition

| Target disease | Development stage | Comments |
|---|---|---|
| AIDS | cell culture | mechanism not fully |
| | | understood |
| Autoimmune | cell culture and animal | high doses necessary |
| diseases | models | |
| Rheumatoid | animal models | |
| Arthritis | | |
| Multiple sclerosis | animal experiments | |
| Psoriasis | cell culture and animal | |
| | experiments | |
| Graft rejection | animal experiments | |
| Wound healing | | |
| Anxiety | effective in animal models | |
| Diabetes type II | Phase II studies | |
| Cancer | cell culture, animal models | DP IV and FAP are |
| | | involved |
| Obesity | animal experiments | NPY, GLP-1 and |
| | | orexine mediated |

### SUMMARY OF THE INVENTION

The inventors of the present application unexpectedly show, that the biodegradation of different substrates, which bind to the same catalytic domain of DP IV and/or DP IV-like enzymes, can be modulated in an unexpected very specific manner.

The invention provides a method to identify the site in the DP IV protein, which is responsible for the modulation of the substrate specificity of DP IV and also provides new compounds, which regulate the substrate specificity of DP IV and which are useful for the treatment of, for example, impaired glucose tolerance, glucosuria, hyperlipidaemia, metabolic acidosis, diabetes mellitus, diabetic neuropathy and nephropathy and of sequelae caused by diabetes mellitus in mammals, metabolism-related hypertension and cardiovascular sequelae caused by hypertension in mammals, for the prophylaxis or treatment of skin diseases and diseases of the mucosae, autoimmune diseases and inflammatory conditions, and for the treatment of psychosomatic, neuropsychiatric and depressive illnesses, such as anxiety, depression, sleep disorders, chronic fatigue, schizophrenia, epilepsy, nutritional disorders, spasm and chronic pain in highly specific manner.

The problem of the invention is solved by using a prolyl oligopeptidase (POP) based computer-generated model of DP IV for the identification of secondary binding sites of DP IV and by providing specific compounds, which bind to at least one secondary binding site and are able to modify very differently the DP IV-catalyzed truncation of substrates of DP IV and DP IV-like enzymes, e.g. bioactive peptides. The overall result is a significant increase of substrate dependent DP IV-selectivity by such compounds and thereby minimization of side reactions with other substrates and as such of potential side effects.

### BRIEF DESCRIPTION OF THE DRAWINGS

Further understanding of these and other aspects of the instant invention may be had by reference to the figures wherein:

FIG. 1 shows a plot of the distribution of the backbone dihedral angles of the complete model of human DP IV. There are no residues in disallowed regions, but some residues are located in only generously allowed areas. Most of them represent residues in loops at the surface of the propeller domain;

FIG. 2 shows the analysis of the quality of the model of human DP IV with regard to some essential stereo-chemical parameters. FIG. 2a expresses the remaining residues not in favored regions, compared to FIG. 1;

FIG. 3 shows the computer-assisted structure model of DP IV and the ADA-binding site (indicated by the arrows and amino acid residue numbers);

FIG. 4 shows the active site of DP IV docked with Ile-Pyr (dark gray);

FIG. 5 shows the interaction of Lys-Z-nitro-pyrrolidine with the active site of DP IV;

FIG. 6 shows the tetrahedral intermediate of Asp-Pro-pNA bound to DP IV;

FIG. 7 shows the interaction of the HIV-tat(1-9) protein with DP IV;

FIG. 8 shows the docking of the N-terminal nonapeptide of the tromboxane receptor;

FIG. 9 shows the 3D-structure model of the interaction between GIP (black thread) and human DP IV;

FIG. 10 shows the docking arrangement of GIP (black) to the active site of DP IV;

FIG. 11(3) shows the molecular dynamic simulation based model of the tertiary structure of GIP (middle part), bound to DP IV. Important amino acid residues from the enzyme are shown in light gray, those from GIP are shown in black, respectively;

FIG. 12 shows the docking of VIP (black) to the active site of DP IV;

FIG. 13 shows the docking of the C-terminal part of VIP to DP IV;

FIG. 14 shows the docking of glucagon (black) to the active site of DP IV;

FIG. (15) shows the molecular dynamic simulation based model of the hexapeptide TFTSDY, bound to the secondary binding site of DP IV. Important amino acid residues from the enzyme are light gray, those from the hexapeptide are marked in dark gray, respectively;

FIG. 16 shows the prolongation of the half-lifes of GIP, Glucagon, PACAP-27 and PACAP-38 by the hexapeptide TFTSDY in a DP IV (porcine and recombinant human) catalyzed peptide truncation test;

FIG. 17 shows the DP IV-catalyzed hydrolysis of RANTES1-15 with (black solid triangle or broken line) or without TFTSDY (black solid square or straight line);

FIG. 18 shows the DP IV-catalyzed hydrolysis of GIP with (black solid triangle) or without TFTSDY (black solid square);

FIG. 19 shows the DP IV-catalyzed hydrolysis of glucagon with (black solid circle) or without TFTSDY (black solid triangle);

FIG. 20 shows a plot of the distribution of the backbone dihedral angles of the complete model of porcine DP IV. All residues are in most favored and additional allowed regions;

FIG. 21 shows the analysis of the quality of the model of porcine DP IV with regard to some essential stereo-chemical parameters of the main chain; and

FIG. 22 shows the analysis of the quality of the model of porcine DP IV with regard to some essential stereo-chemical parameters of the side chains.

### DETAILED DESCRIPTION OF THE INVENTION

The inventors of the present application unexpectedly show, that the biodegradation of substrates, which bind to the same catalytic domain of DP IV, can be modulated very specifically.

One aspect of the invention is to identify the site in the DP IV protein, which is responsible for the modulation of the substrate specificity and selectivity of DP IV and DP IV-like enzymes and to provide new compounds, which regulate the substrate selectivity and/or activity of DP IV and DP IV-like enzymes and which are useful for the treatment of, for example, impaired glucose tolerance, glucosuria, hyperlipidaemia, metabolic acidosis, diabetes mellitus, diabetic neuropathy and nephropathy and of sequelae caused by diabetes mellitus in mammals, metabolism-related hypertension and cardiovascular sequelae caused by hypertension in mammals, for the prophylaxis or treatment of skin diseases and diseases of the mucosae, autoimmune diseases and inflammatory conditions, and for the treatment of psychosomatic, neuropsychiatric and depressive illnesses, such as anxiety, depression, sleep disorders, chronic fatigue, schizophrenia, epilepsy, nutritional disorders, spasm and chronic pain in highly specific manner.

Usually, DP IV is inhibited by compounds mimicking the N-terminal dipeptide part of a DP IV-substrate. This leads to potent compounds which are inhibitors of DP IV and all DP IV-like enzymes and inhibit at sufficient concentrations (e.g. 5×Kᵢ-dose) the DP IV-catalyzed hydrolysis of small chromogenic or higher molecular weight peptide substrates. In the present invention it is demonstrated that compounds interacting with DP IV-binding sites far distant from the catalytic center are capable to differentiate inhibiting the degradation of different substrates, e.g. peptide substrates, or even discriminate DP IV-catalyzed hydrolysis completely.

The substrate properties of the peptides of the growth hormone releasing factor (GRF) family against DP IV were examined.

The GRF family consists of the following peptide hormones:

Gastrin-releasing peptide (GRP)

Enterostatin

Peptide histidine methionine (PHM)

Cholecystokinin

Glucagon-like peptide-2 (GLP-2)

Glucose-dependent insulinotropic polypeptide (GIP)

Glucagon-like peptide-1 (GLP-1)

Growth-hormone releasing factor (GRF)

Pituitary-adenylate cyclase activating polypeptide (PACAP (27 und 38))

Vasoactive intestinale peptide (VIP)

Exendin-1

Exendin-2

Exendin-3

Exendin-4

Secretin

Glucagon

In particular, the capability of purified DP IV from human, from porcine kidney, of recombinant human DP IV and the DP IV activity of the human serum to truncate the peptides of the GRF family were analyzed. The half-life of the peptides were determined using matrix-assisted laser desorption/ionization time-of-flight mass spectrometry (MALDI-TOF MS) whereas the kinetic constants Kₘ and k_{cat}/Kₘ were calculated using capillary zone electrophoresis. All peptides were hydrolyzed by porcine DP IV, recombinant human DP IV or DP IV activity of the human serum. The resulting Kₘ-values were independent from the amino acid in the P₁-position. That means that the binding of substrates to DP IV is not mainly affected by the P₁-residue rather than by secondary interactions between substrate and DP IV protein.

The same surprising phenomenon of different substrate properties was shown with GIP-fragments of different chain lengths. V²GIP(1-6) and G²GIP(1-6) were not hydrolyzed by DP IV. V²GIP(1-30) and G²GIP(1-30) were accepted as substrates and both S²GIP(1-6) and S²GIP(1-30) were truncated by DP IV (Table 1). These findings prove the existence of a secondary binding site in the DP IV protein, which is responsible for substrate recognition and which modulates the biodegradation of substrates and, therefore forms the basis for the management of substrate selectivity and specificity of DP IV.

TABLE 1
Truncation half life of various bioactive peptides which are
substrates for DP IV

| substance | half-life [min] |
|---|---|
| GIP₁₋₃₀ | 2.68 |
| S²GIP₁₋₃₀ | 137.14 |
| V²GIP₁₋₃₀ | 298.04 |
| G²GIP₁₋₃₀ | 150.02 |
| GIP₁₋₆ | <7.5 |
| S²GIP₁₋₆ | 79.04 |
| V²GIP₁₋₆ | no degradation |
| G²GIP₁₋₆ | no degradation |

The amino acid sequences of natural GIP₁₋₃₀ and GIP₁₋₆ are:

GIP₁₋₃₀: YAEGTFISDYSIAMAKIHQQAFVNWLLAQK

GIP₁₋₆: YAEGTF

To identify the secondary binding site, a hexapeptide derived from a consensus sequence of the amino acid sequences of GRF-family peptides was synthesized and its influence on the substrate specificity of DP IV was measured. The selected consensus sequence corresponds to glucagon₅₋₁₀, comprising the amino acid sequence TFTSDY. As expected this peptide had only weak influence on the GP-4-Nitroanilide hydrolysis (K₁=0.71 mM).

In support of the results achieved with the GRF family peptides, the truncation half-lifes of GIP, GLP-1, NPY, glucagon or PACAP by DP IV were also changed after preincubation with 160 μM TFTSDY (Table 2). No differences could be detected between incubation of Rantes₁₋₁₅ and DP IV with or without the hexapeptide TFTSDY (Table 2). The latter finding shows that the peptide Rantes₁₋₁₅ is too short to reach the secondary binding site and therefore TFTSDY has no effect on its hydrolysis rate. The half-lives of GIP and glucagon in presence of DP IV were prolonged by TFTSDY, the strongest influence had TFTSDY on the DP IV-catalyzed truncation of glucagon. Further, a modified variant of the hexapeptide TFTSDY, TFTDDY was synthesized, studied for docking in the DP IV 3D structural model and tested for its regulatory efficacy to modulate substrate specificity of DP IV.

TABLE 2
Inhibitory effect of TFTSDY on DP IV-catalyzed peptide
truncation expressed in Kᵢ-values

K₁ [μM]

| peptide | rec. human DP IV | porcine DP IV |
|---|---|---|
| PACAP-27 | 26.7 | n.d. |
| PACAP-38 | 2.8 | n.d. |
| GIP | 14.0 | 65.9 |
| glucagon | 3.7 | 6.8 |
| RANTES₁₋₁₅ | n.d. | 12307.7 |
| GLP-1 | n.d. | 13.7 |
| NPY | n.d. | 17.2 |

n.d.—not determined

Prolyl oligopeptidase (POP) based computer-generated models of human DP IV and porcine DP IV were used according to the present invention to predict enzyme-substrate-interactions and to identify the interaction site in the DP IV protein structure.

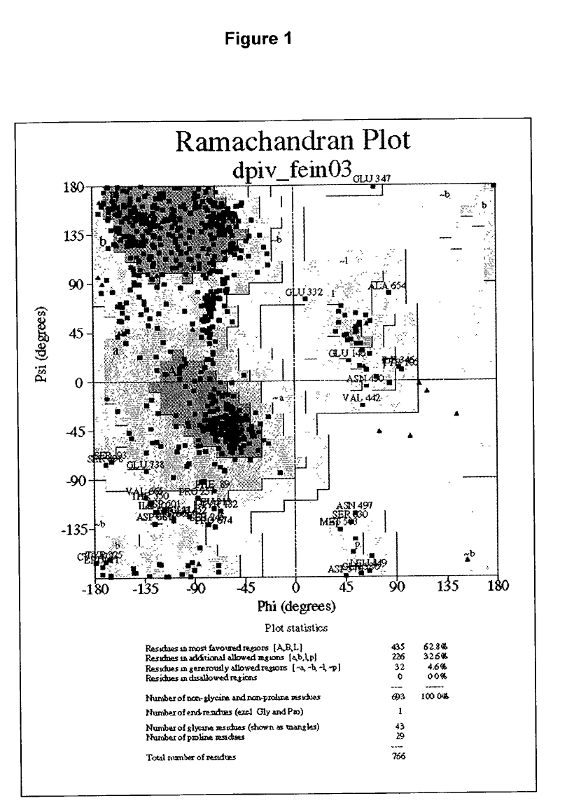

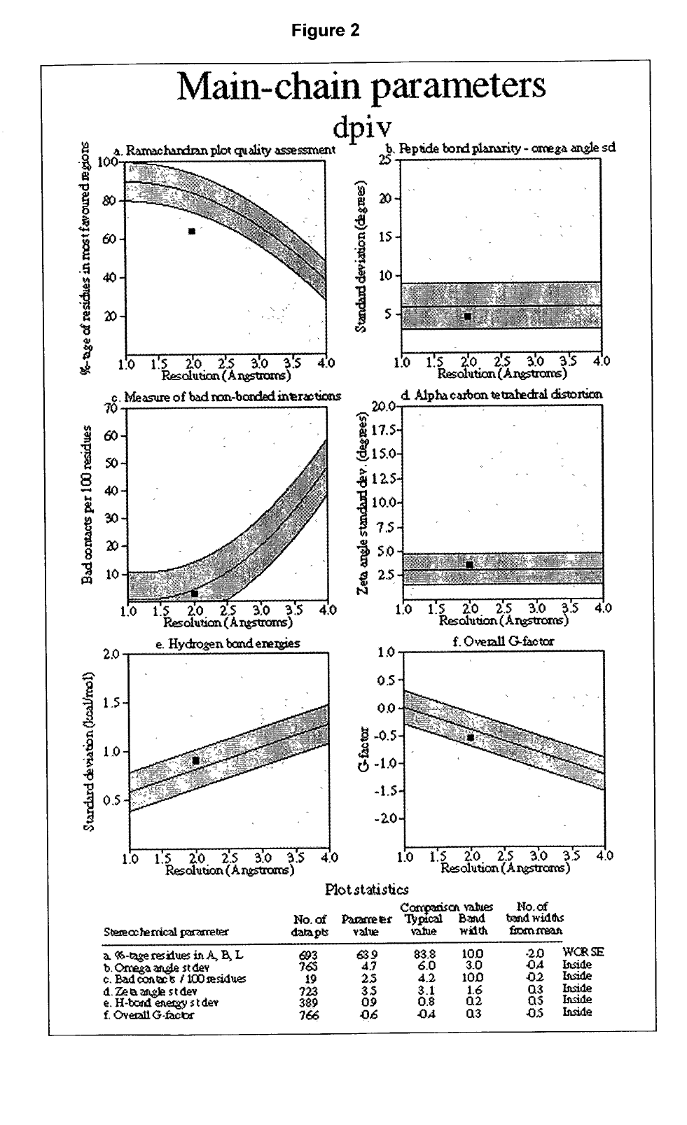

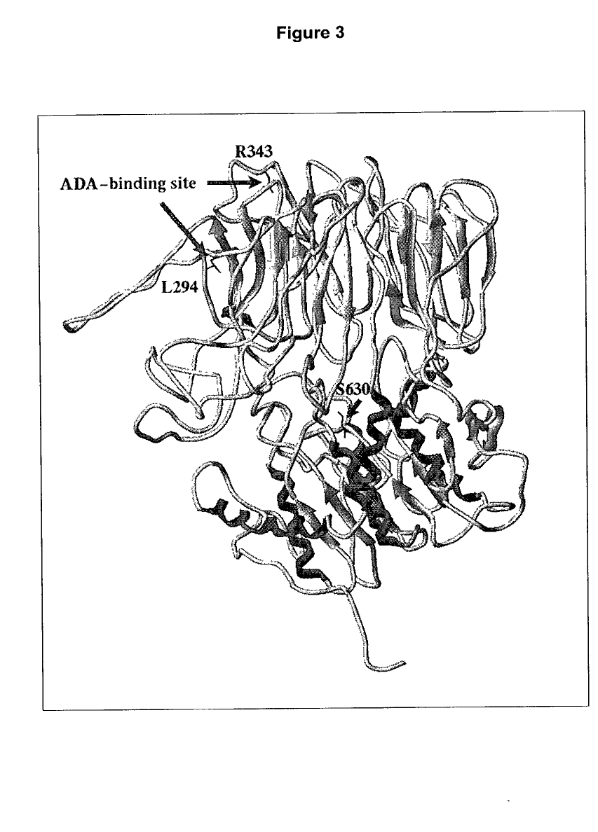

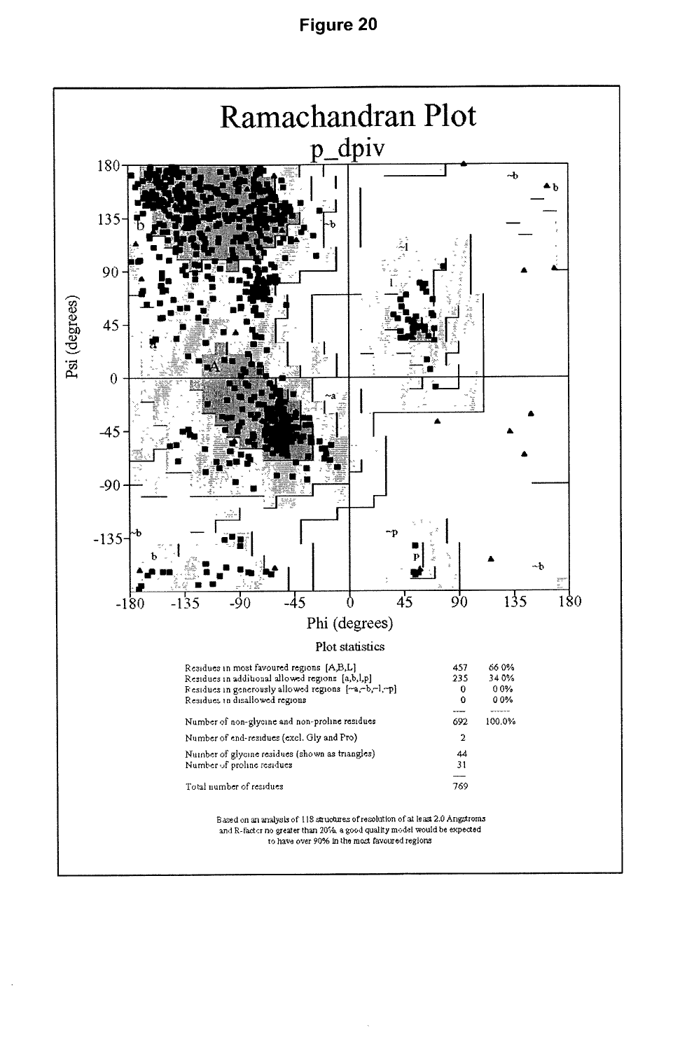

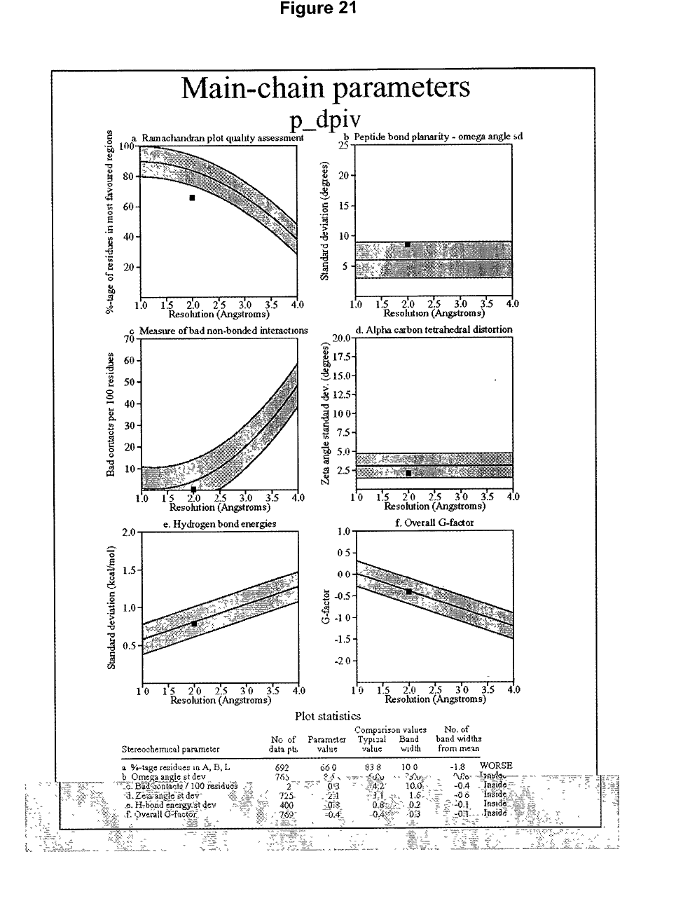

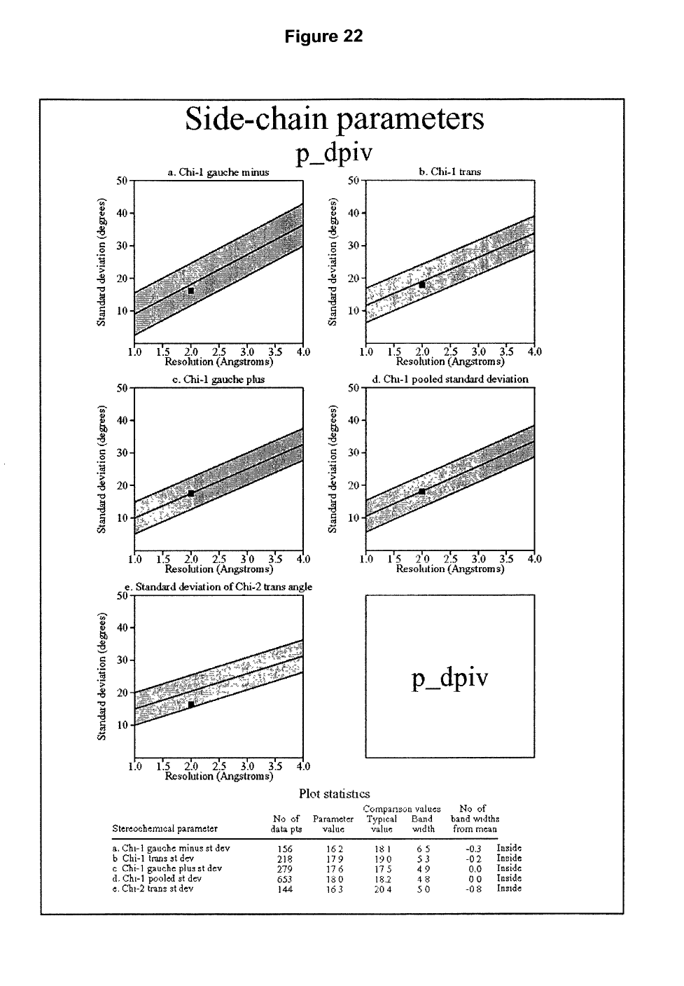

Since the sequence homology between DP IV and the template POP is not very high, standard methods for homology modeling such as the application of COMPOSER gave only very crude preliminary models which needed a lot of manual modification and improvements. These improvements were made by inspection of the conformation and spatial position of each of the 766 amino acid residues with regard to forming sheets or helices and favored intra-residual interactions such as hydrogen bonds, salt bridges and hydrophobic interactions as well. All modifications made were examined by using PROCHECK, which allows the analysis of the stereochemical quality of the model (dihedral angles in favored areas of a Ramachandran Plot, see FIG. 1 for human DP IV and FIG. 20 for porcine DP IV), bond angles and bond length, hydrogen bonds (see FIG. 2 for human DP IV and FIGS. 21 and 22 for porcine DP IV), and by PROSA which analyzes its energy in comparison to native folded proteins. All these residues show that some residues are located in unfavorable areas but all belong to loop regions of the propeller domain which is not of essential importance for docking studies and predictions of new ligands.

In summary of this part, the model of DP IV is now in a state where the overall fold is correct and highly useful for the explanation of experimental results and to allow predictions of recommendations for positions of site directed mutagenesis, development of ligands based on the identified second binding site or selective ligands to bind at the closer active site.

In order to identify essential amino acids for the secondary interaction independently from the active site, site-directed mutageneses were performed using human DP IV cDNA. The mutation sites were: W629A and R560A. The characterization of these mutants showed that both mutations have no influence on the enzyme catalyzed hydrolysis of GP-4-nitroanilide and the kinetic parameters of short and/or low molecular weight inhibitors, which are directed to the active site of DP IV (see table 3). Another mutated enzyme variant, R310A, was expressed as inactive protein. This mutation resulted in the appearance of three DP IV fragments. Based on the computer generated model was shown that an intramolecular salt bridge is formed between R310 and D332 and that this intramolecular salt bridge is crucial for the formation and stabilization of the DP IV tertiary protein structure.

TABLE 3
Kinetic characterization of DP IV-catalyzed substrate hydrolysis by mutants of DP
IV in the secondary binding site

| | Test | Kₘ | Kᵢ | kcat | k_{cat}/Kₘ |
|---|---|---|---|---|---|
| Mutation | compound | [M] | [M] | [s⁻¹] | [M⁻¹*s⁻¹] |
| mu 15 DP IV | Gly-Ser-AMC | Not hydrolyzed | | | |
| mu 15 DP IV | Gly-Pro-AMC | 4.66E−05 | | 1.00E+06 | 2.15E+10 |
| mu 15 DP IV | V²GIP (1-4)* | | no inhibition | | |
| mu 15 DP IV | S²GIP (1-6)* | | no inhibition | | |
| mu 15 DP IV | Glucagon (1-14)* | | no inhibition | | |
| mu 15 DP IV | Leu-Thia-Fum* | | 6.81E−08 | | |
| mu 15 DP IV | TFTSDY* | | no inhibition | | |
| mu 15 DP IV | PACAP (1-38)* | | 3.67E−05 | | |
| mu 15 DP IV | Transp 01* | | 7.69E−08 | | |
| mu 15 DP IV | YAESTF amide* | | 1.14E−06 | | |
| mu 16 DP IV | Gly-Ser-AMC | Not hydrolyzed | | | |
| mu 16 DP IV | Gly-Pro-AMC | 5.02E−05 | | 1.44E+06 | 2.86E+10 |
| mu 16 DP IV | V²GIP (1-4)* | | no inhibition | | |
| mu 16 DP IV | S²GIP (1-6)* | | no inhibition | | |
| mu 16 DP IV | Glucagon (1-14)* | | no inhibition | | |
| mu 16 DP IV | PACAP (1-38)* | | 3.21E−05 | | |
| mu 16 DP IV | Transp 01* | | 8.55E−08 | | |
| mu 16 DP IV | YAESTF amide* | | 1.06E−06 | | |
| mu 16 DP IV | TFTSDY* | | no inhibition | | |
| mu 16 DP IV | Leu-Thia Fum* | | 6.57E−08 | | |
| rh wt DP IV | Gly-Ser-AMC | 4.4E−04 | | | |
| rh wt DP IV | Gly-Pro-AMC | 3.53E−05 | | 1.66E+06 | 4.7E+10 |
| rh wt DP IV | V²GIP (1-4)* | | no inhibition | | |
| rh wt DP IV | S²GIP (1-6)* | | no inhibition | | |
| rh wt DP IV | Glucagon (1-14)* | | no inhibition | | |
| rh wt DP IV | PACAP (1-27)* | 2.28E−04 | 1.13E−04 | | |
| rh wt DP IV | PACAP (1-38)* | | 3.83E−05 | | |
| rh wt DP IV | Transp 01* | | 5.08E−08 | | |
| rh wt DP IV | YAESTF amide* | | 3.51E−08 | | |
| rh wt DP IV | TFTSDY* | | no inhibition | | |
| rh wt DP IV | Leu-Thia Fum* | 4.26E−05 | 6.58E−08 | | |
| p wt DP IV | Leu-Thia-Fum* | 5.98E−05 | 7.29E−08 | | |
| p wt DP IV | PACAP (1-27)* | 1.22E−04 | 5.43E−05 | | |

*The Ki-values were determined in competition of the test compound to the standard substrate GP-4NA (see examples). No inhibition means that the compound doesn't influence the DP IV-catalyzed hydrolysis of the standard substrate GP-4NA.

Definitions in table 3:

| | |
|---|---|
| mu 15 | recombinant human DP IV, mutation R560A |
| mu 16 | recombinant human DP IV, mutation W629A |
| rh wt | recombinant human DP IV, wild type |
| p wt | porcine kidney DP IV, wild type |
| Transp 01 | RRLSYSRRRF-E-Thia |

In the present invention a region was identified in the DP IV-structure, which is responsible for the interaction with a hexapeptide, e.g. TFTSDY. The most important amino acids for the formation of the secondary binding site on DP IV for the GRF family of peptide hormones were found to be but are not restricted to L90, E91, T152, W154, W157, R310, Y330, R318, Y416, S460, K463, E464 and R560.

In the peptides and mutants shown, each encoded residue where appropriate is represented by a one-letter or a three-letter designation, corresponding to the trivial name of the amino acid, in accordance with usual practice. Examples of usual definitions are given the following conventional list:

| Amino Acid | One-Letter Symbol | Three-Letter Symbol |
|---|---|---|
| Alanine | A | Ala |
| Arginine | R | Arg |
| Asparagine | N | Asn |
| Aspartic acid | D | Asp |
| Cysteine | C | Cys |
| Glutamine | Q | Gln |
| Glutamic acid | E | Glu |
| Glycine | G | Gly |
| Histidine | H | His |
| Isoleucine | I | Ile |
| Leucine | L | Leu |
| Lysine | K | Lys |
| Methionine | M | Met |
| Phenylalanine | F | Phe |
| Proline | P | Pro |
| Serine | S | Ser |
| Threonine | T | Thr |
| Tryptophan | W | Trp |
| Tyrosine | Y | Tyr |
| Valine | V | Val |
| Selenocysteine | | Sec |

In a preferred embodiment of the present invention, a secondary binding site in the DP IV protein is identified. More preferred, the existence of this secondary binding site can be used to influence the selectivity of the DP IV-catalyzed biodegradation of DP IV-substrates, e.g. alanine (GIP), proline (GRP) or serine (glucagon) substrates, dependent on the amino acid residue in the P1 position and dependent on the tertiary structure of the DP IV-substrates. Preferred DP IV-substrates, the biodegradation whereof shall be regulated according to the invention with compounds, which bind to the secondary binding site, are serine substrates.

The term “activity modifying” as used in the claims and in the description means both the modification of the enzymatic activity as well as the modification of the selectivity of DP IV and DP IV-like enzymes.

The regulation of the biodegradation of DP IV-substrates due to compounds, which bind to the secondary binding site, is further dependent on the chain length of the substrates. Preferably, DP IV-substrates have a chain length of more than 5 amino acid residues, more preferably more than 10 amino acid residues. Most preferred are substrates with more than 15 amino acid residues up to 70 amino acid residues.

Currently known substrates of DP IV are

Xaa-Pro Peptides

Tyr-melanostatin

Endomorphin-2

Enterostatin

β-Casomorphin

Trypsinogen pro-peptide

Bradykinin

Substance P

Corticotropin-like intermediate lobe peptide

Gastrin-releasing peptide

Neuropeptide Y

Peptide YY

Aprotinin

RANTES

GCP-2

SDF-1α

SDF-1β

MDC

MCP-1

MCP-2

MCP-3

Eotaxin

IP-10

Insulin-like growth factor-I

Pro-colipase

Interleukin-2

Interleukin-1β

α₁-Microglobulin

Prolactin

Trypsinogen

Chorionic Gonadotropin

Xaa-Ala Peptides

PHM

GRH-(1-29)

GRH-(1-44)

GLP-1

GLP-2

Gastric inhibitory peptide

Orexin B

Xaa-Ser Peptides

Orexin A

In the most preferred embodiment of the present invention, compounds for the modulation of DP IV-catalyzed biodegradation of DP IV-substrates are provided, which compounds bind to the secondary binding site of DP IV or DP IV-like enzymes. Such compounds are e.g. selected from the compounds of the formulas a)-d):

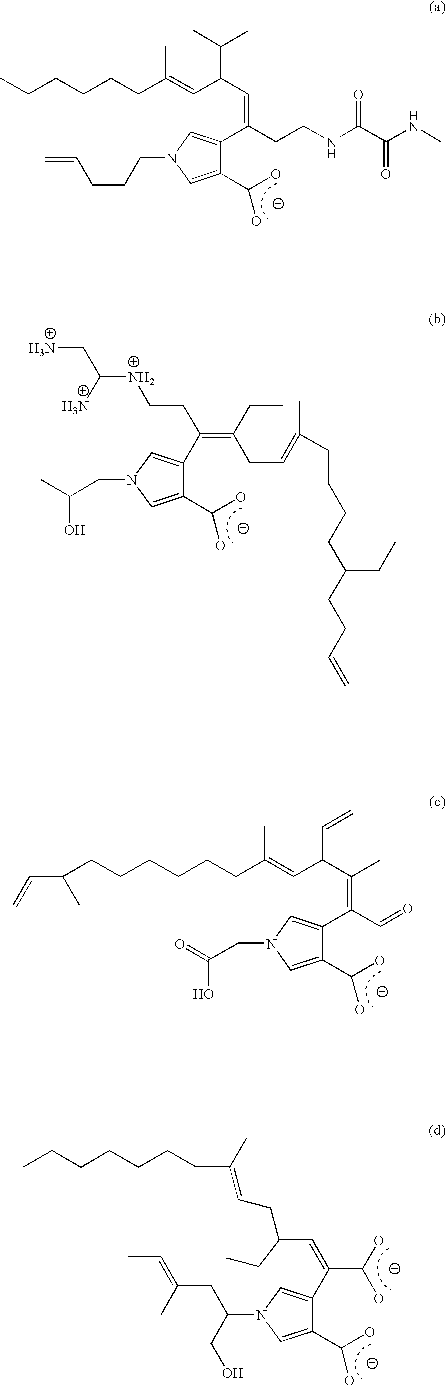

Furthermore, the present invention provides agents, which bind to both the active site and the secondary binding site of DP IV and DP IV-like enzymes and thereby simultaneously modulate the enzyme activity and substrate specificity of DP IV or DP IV-like enzymes.

DP IV is present in a wide variety of mammalian organs and tissues e.g. the intestinal brush-border (Gutschmidt S. et al., “In situ”—measurements of protein contents in the brush border region along rat jejunal villi and their correlations with four enzyme activities. Histochemistry 1981, 72 (3), 467-79), exocrine epithelia, hepatocytes, renal tubuli, endothelia, myofibroblasts (Feller A. C. et al., A monoclonal antibody detecting dipeptidylpeptidase IV in human tissue. Virchows Arch. A. Pathol. Anat. Histopathol. 1986; 409 (2):263-73), nerve cells, lateral membranes of certain surface epithelia, e.g. Fallopian tube, uterus and vesicular gland, in the luminal cytoplasm of e.g., vesicular gland epithelium, and in mucous cells of Brunner's gland (Hartel S. et al., Dipeptidyl peptidase (DPP) IV in rat organs. Comparison of immunohistochemistry and activity histochemistry. Histochemistry 1988; 89 (2): 151-61), reproductive organs, e.g. cauda epididymis and ampulla, seminal vesicles and their secretions (Agrawal & Vanha-Perttula, Dipeptidyl peptidases in bovine reproductive organs and secretions. Int. J. Androl. 1986, 9 (6): 435-52). In human serum, two molecular forms of dipeptidyl peptidase are present (Krepela E. et al., Demonstration of two molecular forms of dipeptidyl peptidase IV in normal human serum. Physiol. Bohemoslov. 1983, 32 (6): 486-96), the serum high molecular weight form of DP IV is expressed on the surface of activated T cells (Duke-Cohan J. S. et al., Serum high molecular weight dipeptidyl peptidase IV (CD26) is similar to a novel antigen DPPT-L released from activated T cells. J. Immunol. 1996, 156 (5): 1714-21). It is also a goal of the present invention to minimize possible side effects of currently available DP IV-inhibitors by the control and management of the DP IV-substrate specificity for the selective treatment of a DP IV mediated disease.

In another preferred embodiment of the present invention, all molecular forms, homologues and epitopes of proteins showing DP IV or DP IV-like enzyme activity, from all mammalian tissues and organs, also of those, which are undiscovered yet, are intended to be embraced by the scope of this invention.

Among the rare group of proline-specific proteases, DP IV was originally believed to be the only membrane-bound enzyme specific for proline as the penultimate residue at the amino-terminus of the polypeptide chain. However, other molecules, even structurally non-homologous with the DP IV but bearing corresponding enzyme activity, have been identified. DP IV-like enzymes, which are identified so far, are e.g. fibroblast activation protein α, dipeptidyl peptidase IV β, dipeptidyl aminopeptidase-like protein, N-acetylated α-linked acidic dipeptidase, quiescent cell proline dipeptidase, dipeptidyl peptidase II, attractin and dipeptidyl peptidase IV related protein (DPP 8), DPL1 (DPX, DP6) and DPL2 are described in the review articles by Sedo & Malik (Sedo & Malik, Dipeptidyl peptidase IV-like molecules: homologous proteins or homologous activities? Biochimica et Biophysica Acta 2001, 36506: 1-10) and Abbott & Gorrell (Abbott, C. A. & Gorrell, M. D., The family of CD26/DP IV and related ectopeptidases. In: Langner & Ansorge (ed.), Ectopeptidases. Kluwer Academic/Plenum Publishers, New York, 2002, pp. 171-195).

Another preferred embodiment of the present invention comprises screening methods for agents which bind to the secondary binding site and/or modulate the selectivity and/or the activity of DP IV or DP IV-like enzymes. An agent according to the invention preferably binds to at least one secondary binding site of the DP IV or DP IV-like enzyme proteins.

The screening method for agents of the secondary binding site comprises the following steps:

a) Contacting said compounds with DP IV or a DP IV-like enzyme, preferably under conditions which would permit binding therebetween;

b) Adding a substrate of DP IV or DP IV-like enzymes to said DP IV or DP IV-like enzyme;

c) Monitoring the biodegradation of the substrate or optionally measuring the residual DP IV or DP IV-like enzyme activity; and

d) Correlating changes in the biodegradation and/or enzyme activity with the binding of said compounds to DP IV or DP IV-like enzymes to identify a selectivity and/or activity modifying agent.

The agents (compounds) selected by the above described screening method can work by regulating (increasing or decreasing) the biodegradation of at least one substrate of DP IV or the DP IV-like enzyme, preferably by the prolongation of the half-life of such substrate, most preferably by the inhibition of the biodegradation of such substrate.

Conditions, under which binding between compounds and DP IV or DP IV-like enzymes are permitted, are described, e.g. in example 2.

DP IV or DP IV-like enzymes as used in the screening method described above mean purified DP IV or DP IV-like enzymes from mammals, selected from but not restricted to human, monkey, mouse, rat etc., or DP IV or DP IV-like enzyme containing cells and cell lines from mammals, selected from but not restricted to human, monkey, mouse, rat etc., or DP IV or DP IV-like enzyme containing cell extracts or body liquids e.g. liver extracts, blood plasma samples, blood serum samples, brain extracts etc., from such mammals.

Preferably, an agent increases the selectivity and/or activity of DP IV or DP IV-like enzymes towards substrates by at least about 10, preferably about 50, more preferably about 75, 90 or 100% relative to the absence of the agent. More preferably, an agent increases the selectivity and/or activity of DP IV or DP IV-like enzymes towards specific substrates by at least about 10, preferably about 50, more preferably about 75, 90 or 100% and prolongs the half live of the substrates in the serum or in the plasma of a mammal at least about 1fold, preferably about 2fold, more preferably about 3fold, 4fold or higher relative to the absence of the agent. Most preferably, an agent increases the selectivity and/or activity of DP IV or DP IV-like enzymes in such a way that the half live of at least one substrate in the serum or in the plasma of a mammal is increased at least about 1fold, preferably about 2fold, more preferably about 3fold, 4fold or higher, most preferably complete inhibition of the degradation of such a substrate is achieved, relative to the absence of the agent.

It is also preferred according to the invention that the agents modulate the interaction between DP IV or DP IV-like enzymes and binding proteins thereof. Binding proteins are proteins that bind other proteins in a non-covalent manner and thereby modulate their activity or serve as carriers of these proteins. Binding proteins of DP IV (CD26) identified so far include adenosine deaminase, two proteins of HIV, transactivator protein (tat) and the gp120 envelope protein, CD45, a membrane located tyrosine phosphatase, extracellular matrix proteins, such as collagen and fibronectin, plasminogen and streptokinase, mannose 6-phosphat/insulin-like growth factor II receptor, the isoform NH3 of the Na⁺/H⁺ exchanger from renal microvilly membranes and the thromboxane A2 receptor.

Agents (also called compounds herein) can be pharmacological agents already known in the art or can be compounds previously unknown to have any pharmacological activity. The compounds can be naturally occurring or designed in the laboratory. They can be isolated from microorganisms, animals, or plants, and can be produced recombinantly, or synthesized by chemical methods in the art. If desired, agents can be obtained using any of the numerous combinatorial library methods known in the art, including but not limited to, biological libraries, spatially addressable parallel solid phase or solution phase libraries, synthetic library methods requiring deconvolution, the “one-bead-one-compound” library method, and synthetic library methods using affinity chromatography selection. The biological library approach is limited to polypeptide libraries, while the other four approaches are applicable to polypeptide, non-peptide oligomer, or small molecule libraries of compounds. See Lam, Anticancer Drug Des., 12, 145, 1997.

Methods for the synthesis of molecular libraries are well known in the art (see, for example, De Witt et al., Proc. Natl. Acad. Sci. USA 90, 6909, 1993; Erb et al. Proc. Natl. Acad. Sci. USA 91, 11422, 1994; Zuckermann et al., J. Med. Chem. 37, 2678, 1994; Cho et al., Science 261, 1303, 1993; Carell et al., Angew. Chem. Int. Ed. Engl. 33, 2059, 1994; Carell et al., Angew. Chem. Int. ed. engl. 33, 2061; Gallop et al., J. Med. Chem. 37, 1233, 1994). Libraries of compounds can be present in solution (see, e.g. Houghten, Bio Techniques 13, 412421, 1992) or on beads (Lam, nature 354, 824, 1991) chips (Fodor, Nature 364, 555556, 1993) bacteria or spores (Ladner, U.S. Pat. No. 5,223,409), plasmids (Cull et al., Proc. Natl. Acad. Sci. USA 89, 198651869, 1992), or phage (Scott & Smith, Science 249, 386390, 1990; Devlin, Science 249, 404406, 1990); Cwirla et la., Proc. Natl. Acad. Sci. 97, 63786382, 1990; Felici, J. Mol. Biol. 222, 301310, 1991; and Ladner, U.S. Pat. No. 5,223,409).

High Throughput Screening

Agents can be screened for the ability to bind to DP IV or DP IV-like enzymes or to affect DP IV or DP IV-like enzyme activity using high throughput screening. Using high throughput screening, many discrete compounds can be tested in parallel so that large numbers of agents can be quickly screened. The most widely established techniques utilize 96-well microtiter plates. The well of the microtiter plates typically require assay volumes that range from 50 to 500 μl. In addition to the plates, many instruments, materials, pipettors, robotics, plate washers, and plate readers are commercially available to fit the 96-well format.

Alternatively, “Free format assays”, or assays that have no physical barrier between samples, can be used. For example, an assay using pigment cells (melanocytes) in a simple homogeneous assay for combinatorial peptide libraries is described by Jayawickreme et al., Proc. Natl. Acad. Sci. USA 19, 161418 (1994).

Another example of a free format assay is described by Chelsky, “Strategies for Screening Combinatorial Libraries: Novel and Traditional Approaches,” reported at the First Annual conference of The Society for Biomolecular Screening in Philadelphia, Pa. (November 710, 1995). Chelsky placed a simple homogenous enzyme assay for carbonic anhydrase inside an agarose gel such that the enzyme in the gel would cause a color change throughout the gel. Thereafter, beads carrying combinatorial compounds were partially released by UV LIGHT. Compounds that inhibited the enzyme were observed as local zones of inhibition having less color change.

Yet another example is described by Salomon et al., Molecular Diversity 2, 5763 (1996). In this example, combinatorial libraries were screened for compounds that had cytotoxic effects on cancer cells growing in agar.

Another high throughput screening method is described in Beutel et al., U.S. Pat. No. 5,976,813. In this method, test samples are placed in a porous matrix. One or more assay components are then placed within, on top of, or at the bottom of a matrix such as a gel, a plastic sheet, a filter, or other form of easily manipulated solid support. When samples are introduced to the porous matrix they diffuse sufficiently slowly, such that the assays can be performed without the test samples running together.

Binding Assays

For binding assays, the agent is preferably a small molecule which binds to and occupies, the secondary binding site of DP IV or DP IV-like enzymes, such that normal biological activity is changed or prevented. Examples of such small molecules include, but are not limited to, small peptides or peptide like molecules.

In binding assays, either the agent of DP IV or the DP IV-like enzyme can comprise a detectable label, such as a fluorescent, radioisotopic, chemiluminescent, or the enzyme is labeled, such as horseradish peroxidase, alkaline phosphatase, or luciferase. Detection of an agent, which is bound to DP IV or the DP IV-like enzyme can then be accomplished, for example, by direct counting of radioemmission, by scintillation counting, or by determining conversion of an appropriate substrate to a detectable product.

Alternatively, binding of an agent to DP IV or a DP IV-like enzyme can be determined without labelling either of the interactants. For example, a microphysiometer can be used to detect binding of an agent with DP IV or a DP IV-like enzyme. A microphysiometer (e.g., Cytosensor™) is an analytical instrument that measures the rate at which a cell acidifies its environment using a light-addressable potentiometric sensor (LAPS). Changes in this acidification rate can be used as an indicator of the interaction between an agent and DP IV or a DP IV-like enzyme (McConnel et al., Science 257, 19061912, 1992).

Determining the ability of an agent to bind to DP IV or a DP IV-like enzyme also can be accomplished using a technology such as real-time Biomolecular Interaction Analysis (BIA) (Sjolander & Urbaniczky, Anal. Chem. 63, 23382345, 1991, and Szabo et al., Curr. Opin. Struct. Biol. 5, 699705, 1995) BIA is a technology for studying biospecific interactions in real time, without labelling any of the interactants (e.g. BIAcore™). Changes in the optical phenomenon surface plasmon resonance (SPR) can be used as an indication of real-time reactions between biological molecules.

In yet another aspect of the invention, DP IV or a DP IV-like enzyme can be used as a “bait protein” in a two hybrid assay or three-hybrid assay (see, e.g. U.S. Pat. No. 5,283,317; Zervos et al., Cell 72, 223232, 1993; Madura 920924, 193; Iwabuchi et al., Oncogene 8, 16931696, 1993; and Brent WO94/10300), to identify other proteins which bind to or interact with the DP IV or the DP IV-like enzyme and modulate its activity.

The two hybrid system is based on the modular nature of most transcription factors, which consist of separable DNA binding and activation domains. Briefly, the assay utilizes two different DNA constructs. For example, in one construct, a polynucleotide encoding DP IV or a DP IV-like enzyme can be fused to a polynucleotide encoding the DNA binding domain of a known transcription factor (e.g. GAL4). In the other construct a DNA sequence that encodes an unidentified protein (“prey” or “sample”) can be fused to a polynucleotide that codes for the activation domain of the known transcription factor. If the “bait” and the “prey” proteins are able to interact in vivo to form an protein dependent complex, the DNA binding and activation domains of the transcription factor are brought into close proximity. This proximity allows transcription of a reporter gene (e.g. LacZ), which is operably linked to a transcriptional regulatory site responsive to the transcription factor. Expression of the reporter gene can be detected, and cell colonies containing the functional transcription factor can be isolated and used to obtain the DNA sequence enconding the protein which interacts with the dipeptidyl-peptidase IV-like enzyme polypeptide.

It may be desirable to immobilize either the DP IV or DP IV-like enzyme or the agent to facilitate separation of bound from unbound forms of one or both of the interactants, as-well-as to accommodate automation of the assay. Thus, either DP IV or the DP IV-like enzyme or the agent can be bound to a solid support. Suitable solid supports include, but are not limited to, glass or plastic slices, tissue culture plates, microtiter wells, tubes, silicon chips, or particles such as beads (including, but not limited to latex, polysterene, or glass beads). Any method known in the art can be used to attach DP IV or the DP IV-like enzyme or agent to a solid support, including use of covalent and non-covalent linkages, passive absorption, or pairs of binding moieties attached respectively to the polypeptide or agent and the solid support. Agents are preferably bound to the solid support in an array, so that the location of individual test compounds can be tracked. Binding of a test compound to a DP IV or a DP IV-like enzyme can be accomplished in any vessel suitable for containing the reactants. Examples of such vessels include microtiter plates, test tubes, and microcentrifuge tubes.

In one embodiment, the DP IV or DP IV-like enzyme is a fusion protein comprising a domain that allows the DP IV or DP IV-like enzyme to be bound to a solid support. For example, glutathione-S-transferase fusion proteins can be absorbed onto glutathione sepharose beads (Sigma Chemical, St. Louis, Mo.) or glutathione derivatized microtiter plates, which are then combined with the agent and the non-absorbed DP IV or DP IV-like enzyme; the mixture is then incubated under conditions conducive to complex formation (e.g., at physiological conditions for salt and pH). Following incubation, the beads or microtiter plate wells are washed to remove any unbound components. Binding of the interactants can be determined either directly or indirectly, as described above. Alternatively, the complexes can be dissociated from the solid support before binding is determined.

Other techniques for immobilizing proteins on a solid support also can be used in the screening assays of the invention. For example, either DP IV or a DP IV-like enzyme or an agent can be immobilized utilizing conjugation of biotin and streptavidin. Biotinylated DP IV or DP IV-like enzymes or agents can be prepared from biotin-NHS-(N-hydroxysuccinimide) using techniques well known in the art (e.g. biotinylation kit, Pierce Chemicals, Rockford, Ill.) and immobilized in the wells of streptavidin-coated 96 well plates (Pierce chemical). Alternatively, antibodies which specifically bind to DP IV, a DP IV-like enzyme or an agent, but which do not interfere with a desired binding site, such as secondary binding site or the active site of DP IV or the DP IV-like enzyme, can be derivatized to the wells of the plate. Unbound targets or proteins can be trapped in the wells by antibody conjugation.

Examples for commercial available antibodies against DP IV or CD26 are for instance:

| | | Species | | |
|---|---|---|---|---|
| Company | Clone | (antigen) | Application | Host |
| Coulter | Ta1 | human | IF, FACS | Mouse |
| | Ba5 | human | FACS | |
| Biozol | TA59 (Endogen) | human | ICH* | |
| Pharmingen | M-A216 | human | IF, FACS | Mouse |
| Biotrend | 13.4 | rat | ICH, IF | Mouse |
| | M-T099 | human | ICH, IF | Mouse |
| | 134-2C2 | human | IF, FACS | Mouse |
| | LT-27 | human | IF, FACS | Mouse |
| Biozol | MRCOX-61 | rat | FC | Mouse |
| Biozol | 236.3 | rat | IF, IPrep, | Mouse |
| | | | IHstaining | |
| Research | 202.36 | human | IF | Mouse |
| Diagnostics | | | | |
| Research | 134-2C2 | human | T-cell | Mouse |
| Diagnostics | | | signaling, HIV | |
| | | | infection | |

Methods for detecting such complexes, in addition to those described above for the GST-immobilized complexes, include immunodetection of complexes using antibodies which specifically bind to DP IV or DP IV-like enzymes or the agent, enzyme linked assays which rely on detecting an activity of the DP IV or the DP IV-like enzyme, and SDS gel electrophoresis under non-reducing conditions.

Screening for agents which bind to DP IV or a DP IV-like enzyme also can be carried out in an intact cell. Any cell which comprises DP IV or a DP IV-like enzyme can be used in a cell-based assay system. DP IV or a DP IV-like enzyme can be naturally occurring in the cell or can be introduced using techniques such as those described above. Binding of the agents to DP IV or a DP IV-like enzyme is determined as described above.

Enzyme Assays

Agents can be tested for the ability to increase or decrease the activity of a mamalien DP IV or DP IV-like enzyme. DP IV activity can be measured, for example, as described in U.S. Pat. No. 5,601,986 and, specific for the present invention, in examples 1 to 3.

Further on, a screening method for the identification and determination of one or more secondary binding sites on DP IV and DP IV-like enzymes is provided.

The screening method for secondary binding site(s) of DP IV and DP IV-like enzymes comprises the following steps:

a) providing two or more substrates, having an amino acid sequence, binding to DP IV or DP IV-like enzymes and aligning the amino acid sequences of said substrates;

b) identifying at least one consensus sequence amongst said substrate amino acid sequences;

c) synthesizing a peptide having such a consensus sequence;

d) contacting said synthesized peptide with DP IV or a DP IV-like enzyme;

e) adding a substrate of DP IV or DP IV-like enzymes to the DP IV or DP IV-like enzyme;

f) monitoring the biodegradation of the substrate or optionally measuring the residual DP IV or DP IV-like enzyme activity; and

g) correlating changes in said biodegradation or enzyme activity with the presence of a second binding site capable of modulating the substrate specificity of DP IV or DP IV-like enzymes.

1. Consensus sequences are highly conserved sequence segments. Preferred according to the invention are consensus sequences with the length of 3 to 20 amino acids, more preferred of 5 to 12 amino acids, most preferred 5 to 7 amino acids.

In another illustrative embodiment of the present invention, the agents, which bind to the secondary binding site, e.g. obtained or selected by the screening method described herein, can be used alone or in combination with DP IV-inhibitors for the treatment of any type of DP IV mediated disorders, selected but not restricted to, impaired glucose tolerance, glucosuria, hyperlipidaemia, metabolic acidosis, diabetes mellitus, diabetic neuropathy and nephropathy and of sequelae caused by diabetes mellitus in mammals, metabolism-related hypertension and cardiovascular sequelae caused by hypertension in mammals, for the prophylaxis or treatment of skin diseases and diseases of the mucosae, autoimmune diseases and inflammatory conditions, and for the treatment of psychosomatic, neuropsychiatric and depressive illnesses, such as anxiety, depression, sleep disorders, chronic fatigue, schizophrenia, epilepsy, nutritional disorders, spasm and chronic pain.

Agents such as N-(N′-substituted glycyl)-2-cyanopyrrolidines, L-threo-isoleucyl thiazolidine (P32/98), L-allo-isoleucyl thiazolidine, L-threo-isoleucyl pyrrolidine, and L-allo-isoleucyl pyrrolidine have been developed which inhibit the enzymatic activity of DP IV and are described in U.S. Pat. No. 6,001,155, WO 99/61431, WO 99/67278, WO 99/67279, DE 198 34 591, WO 97/40832, DE 196 16 486 C 2, WO 98/19998, WO 00/07617, WO 99/38501, and WO 99/46272. Further examples of low molecular weight dipeptidyl peptidase IV inhibitors are agents such as tetrahydroisoquinolin-3-carboxamide derivatives, N-substituted 2-cyanopyroles and—pyrrolidines, N-(N′-substituted glycyl)-2-cyanopyrrolidines, N-(substituted glycyl)-thiazolidines, N-(substituted glycyl)-4-cyanothiazoidines, amino-acyl-borono-prolyl-inhibitors and cyclopropyl-fused pyrrolidines. Inhibitors of dipeptidyl peptidase IV are described in U.S. Pat. No. 6,011,155; U.S. Pat. No. 6,107,317; U.S. Pat. No. 6,110,949; U.S. Pat. No. 6,124,305; U.S. Pat. No. 6,172,081; WO 99/61431, WO 99/67278, WO 99/67279, DE 198 34 591, WO 97/40832, DE 196 16 486 C 2, WO 98/19998, WO 00/07617, WO 99/38501, WO 99/46272, WO 99/38501, WO 01/68603, WO 01/40180, WO 01/81337, WO 01/81304, WO 01/55105, WO 02/02560, WO 02/14271 and WO 02/051836, the teachings of which are herein incorporated by reference in their entirety concerning these inhibitors, their uses, definition and their production. The goal of these agents is to inhibit DP IV, and by doing so, to relieve effectively any type of DP IV-mediated disease. The inventors hereof have surprisingly discovered that such agents can be advantageously employed for an entirely different therapeutic purpose, then previously known by those skilled in the art.

Preferred for the use in combination with agents binding to the secondary binding site of DP IV or DP IV-like enzymes are DP IV-inhibitors such as NVP-DPP728A (1-[[[2-[{5-cyanopyridin-2-yl}amino]ethyl]amino]acetyl]-2-cyano-(S)-pyrrolidine) (Novartis) as disclosed by Hughes et al., Biochemistry, 38 (36), 11597-11603, 1999, LAF-237 (1-[(3-hydroxy-adamant-1-ylamino)-acetyl]-pyrrolidine-2(S)-carbonitrile); disclosed by Hughes et al., Meeting of the American Diabetes Association 2002, Abstract no. 272 or (Novartis), TSL-225 (tryptophyl-1,2,3,4-tetrahydroisoquinoline-3-carboxylic acid), disclosed by Yamada et. al., Bioorg. & Med. Chem. Lett. 8 (1998), 1537-1540, 2-cyanopyrrolidides and 4-cyanopyrrolidides as disclosed by Asworth et al., Bioorg. & Med. Chem. Lett., 6, No. 22, pp 1163-1166 and 2745-2748 (1996), FE-999011, disclosed by Sudre et al., Diabetes 51 (5), pp 1461-1469 (2002) (Ferring) and the compounds disclosed in WO 01/34594 (Guilford), employing dosages as set out in the above references.

In one especially illustrative embodiment, the present invention relates to the use of agents, which bind to the secondary binding site(s) of DP IV or DP IV-like enzymes in combination with dipeptide-like compounds and compounds analogous to dipeptide compounds that are formed from an amino acid and a thiazolidine or pyrrolidine group, and salts thereof, referred to hereinafter as dipeptide-like compounds. Preferably the amino acid and the thiazolidine or pyrrolidine group are bonded with an amide bond.

Especially suitable for that purpose according to the invention are dipeptide-like compounds in which the amino acid is preferably selected from a natural amino acid, such as, for example, leucine, valine, glutamine, glutamic acid, proline, isoleucine, asparagines and aspartic acid.

The dipeptide-like compounds used according to the invention exhibit at a concentration (of dipeptide compounds) of 10 μM, a reduction in the activity of plasma dipeptidyl peptidase IV or DP IV-analogous enzyme activities of at least 10%, especially of at least 40%. Frequently a reduction in activity of at least 60% or at least 70% is also required. Preferred agents may also exhibit a reduction in activity of a maximum of 20% or 30%.

Preferred compounds are N-valyl prolyl, O-benzoyl hydroxylamine, alanyl pyrrolidine, isoleucyl thiazolidine like L-allo-isoleucyl thiazolidine, L-threo-isoleucyl pyrrolidine and salts thereof, especially the fumaric salts, and L-allo-isoleucyl pyrrolidine and salts thereof. Especially preferred compounds are glutaminyl pyrrolidine and glutaminyl thiazolidine of formulas 1 and 2:

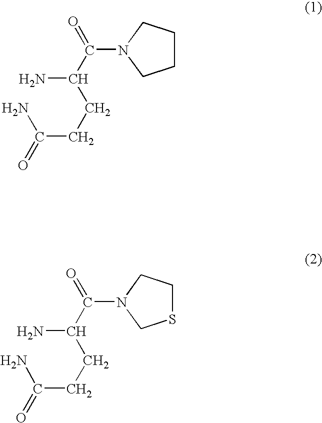

Further preferred compounds are given in Table 4.

The salts of the dipeptide-like compounds can be present in a molar ratio of dipeptide (-analogous) component to salt component of 1:1 or 2:1. Such a salt is, for example, (Ile-Thia)₂ fumaric acid.

TABLE 4
Structures of further preferred dipeptide compounds

DP IV-inhibitor
H-Asn-pyrrolidine
H-Asn-thiazolidine
H-Asp-pyrrolidine
H-Asp-thiazolidine
H-Asp(NHOH)-pyrrolidine
H-Asp(NHOH)-thiazolidine
H-Glu-pyrrolidine
H-Glu-thiazolidine
H-Glu(NHOH)-pyrrolidine
H-Glu(NHOH)-thiazolidine
H-His-pyrrolidine
H-His-thiazolidine
H-Pro-pyrrolidine
H-Pro-thiazolidine
H-Ile-azididine
H-Ile-pyrrolidine
H-L-allo-Ile-thiazolidine
H-Val-pyrrolidine
H-Val-thiazolidine

In another preferred embodiment, the present invention provides the use of agents binding to the secondary binding site(s) of DP IV or DP IV-like enzymes in combination with substrate-like peptide compounds of formula 3 useful for competitive modulation of dipeptidyl peptidase IV catalysis:

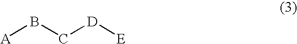

wherein

A, B, C, D and E are independently any amino acid moieties including proteinogenic amino acids, non-proteinogenic amino acids, L-amino acids and D-amino acids and wherein E and/or D may be absent.

Further conditions regarding formula (3):

A is an amino acid except a D-amino acid,

B is an amino acid selected from Pro, Ala, Ser, Gly, Hyp, acetidine-(2)-carboxylic acid and pipecolic acid,

C is any amino acid except Pro, Hyp, acetidine-(2)-carboxylic acid, pipecolic acid and except N-alkylated amino acids, e.g. N-methyl valine and sarcosine,

D is any amino acid or missing, and

E is any amino acid or missing,

or:

C is any amino acid except Pro, Hyp, acetidine-(2)-carboxylic acid, pipecolic acid, except N-alkylated amino acids, e.g. N-methyl valine and sarcosine, and except a D-amino-acid;

D is any amino acid selected from Pro, Ala, Ser, Gly, Hyp, acetidine-(2)-carboxylic acid and pipecolic acid, and

E is any amino acid except Pro, Hyp, acetidine-(2)-carboxylic acid, pipecolic acid and except N-alkylated amino acids, e.g. N-methyl valine and sarcosine.

Examples of amino acids which can be used in the present invention are: L and D-amino acids, N-methyl-amino-acids; allo- and threo-forms of Ile and Thr, which can, e.g. be α-, β- or ω-amino acids, whereof α-amino acids are preferred.

Examples of amino acids throughout the claims and the description are: aspartic acid (Asp), glutamic acid (Glu), arginine (Arg), lysine (Lys), histidine (His), glycine (Gly), serine (Ser) and cysteine (Cys), threonine (Thr), asparagine (Asn), glutamine (Gln), tyrosine (Tyr), alanine (Ala), proline (Pro), valine (Val), isoleucine (Ile), leucine (Leu), methionine (Met), phenylalanine (Phe), tryptophan (Trp), hydroxyproline (Hyp), beta-alanine (beta-Ala), 2-amino octanoic acid (Aoa), azetidine-(2)-carboxylic acid (Ace), pipecolic acid (Pip), 3-amino propionic, 4-amino butyric and so forth, alpha-aminoisobutyric acid (Aib), sarcosine (Sar), ornithine (Orn), citrulline (Cit), homoarginine (Har), t-butylalanine (t-butyl-Ala), t-butylglycine (t-butyl-Gly), N-methylisoleucine (N-MeIle), phenylglycine (Phg), cyclohexylalanine (Cha), norleucine (Nle), cysteic acid (Cya) and methionine sulfoxide (MSO), Acetyl-Lys, modified amino acids such as phosphoryl-serine (Ser(P)), benzyl-serine (Ser(Bzl)) and phosphoryl-tyrosine (Tyr(P)), 2-aminobutyric acid (Abu), aminoethylcysteine (AECys), carboxymethylcysteine (Cmc), dehydroalanine (Dha), dehydroamino-2-butyric acid (Dhb), carboxyglutaminic acid (Gla), homoserine (Hse), hydroxylysine (Hyl), cis-hydroxyproline (cisHyp), trans-hydroxyproline (transHyp), isovaline (Iva), pyroglutamic acid (Pyr), norvaline (Nva), 2-aminobenzoic acid (2-Abz), 3-aminobenzoic acid (3-Abz), 4-aminobenzoic acid (4-Abz), 4-(aminomethyl)benzoic acid (Amb), 4-(aminomethyl)cyclohexanecarboxylic acid (4-Amc), Penicillamine (Pen), 2-Amino-4-cyanobutyric acid (Cba), cycloalkane-carboxylic aicds.

Examples of ω-amino acids are e.g.: 5-Ara (aminoraleric acid), 6-Ahx (aminohexanoic acid), 8-Aoc (aminooctanoic aicd), 9-Anc (aminovanoic aicd), 10-Adc (aminodecanoic acid), 11-Aun (aminoundecanoic acid), 12-Ado (aminododecanoic acid).

Further amino acids are: indanylglycine (Igl), indoline-2-carboxylic acid (Idc), octahydroindole-2-carboxylic acid (Oic), diaminopropionic acid (Dpr), diaminobutyric acid (Dbu), naphtylalanine (1-Nal), (2-Nal), 4-aminophenylalanin (Phe(4-NH₂)), 4-benzoylphenylalanine (Bpa), diphenylalanine (Dip), 4-bromophenylalanine (Phe(4-Br)), 2-chlorophenylalanine (Phe(2-Cl)), 3-chlorophenylalanine (Phe(3-Cl)), 4-chlorophenylalanine (Phe(4-Cl)), 3,4-chlorophenylalanine (Phe (3,4-Cl₂)), 3-fluorophenylalanine (Phe(3-F)), 4-fluorophenylalanine (Phe(4-F)), 3,4-fluorophenylalanine (Phe(3,4-F₂)), pentafluorophenylalanine (Phe(F₅)), 4-guanidinophenylalanine (Phe(4-guanidino)), homophenylalanine (hPhe), 3-jodophenylalanine (Phe(3-J)), 4 jodophenylalanine (Phe(4-J)), 4-methylphenylalanine (Phe(4-Me)), 4-nitrophenylalanine (Phe-4-NO₂)), biphenylalanine (Bip), 4-phosphonomehtylphenylalanine (Pmp), cyclohexyglycine (Ghg), 3-pyridinylalanine (3-Pal), 4-pyridinylalanine (4-Pal), 3,4-dehydroproline (A-Pro), 4-ketoproline (Pro(4-keto)), thioproline (Thz), isonipecotic acid (Inp), 1,2,3,4,-tetrahydroisoquinolin-3-carboxylic acid (Tic), propargylglycine (Pra), 6-hydroxynorleucine (NU(6-OH)), homotyrosine (hTyr), 3-jodotyrosine (Tyr(3-J)), 3,5-dijodotyrosine (Tyr(3,5-J₂)), d-methyl-tyrosine (Tyr(Me)), 3-NO₂-tyrosine (Tyr(3-NO₂)), phosphotyrosine (Tyr(PO₃H₂)), alkylglycine, 1-aminoindane-1-carboxy acid, 2-aminoindane-2-carboxy acid (Aic), 4-amino-methylpyrrol-2-carboxylic acid (Py), 4-amino-pyrrolidine-2-carboxylic acid (Abpc), 2-aminotetraline-2-carboxylic acid (Atc), diaminoacetic acid (Gly(NH₂)), diaminobutyric acid (Dab), 1,3-dihydro-2H-isoinole-carboxylic acid (Disc), homocylcohexylalanin (hCha), homophenylalanin (hPhe oder Hof), trans-3-phenyl-azetidine-2-carboxylic acid, 4-phenyl-pyrrolidine-2-carboxylic acid, 5-phenyl-pyrrolidine-2-carboxylic acid, 3-pyridylalanine (3-Pya), 4-pyridylalanine (4-Pya), styrylalanine, tetrahydroisoquinoline-1-carboxylic acid (Tiq), 1,2,3,4-tetrahydronorharmane-3-carboxylic acid (Tpi), β-(2-thienryl)-alanine (Tha).

Other amino acid substitutions for those encoded in the genetic code can also be included in peptide compounds within the scope of the invention and can be classified within this general scheme.

Proteinogenic amino acids are defined as natural protein-derived α-amino acids. Non-proteinogenic amino acids are defined as all other amino acids, which are not building blocks of common natural proteins.

The resulting peptides may be synthesized as the free C-terminal acid or as the C-terminal amide form. The free acid peptides or the amides may be varied by side chain modifications. Such side chain modifications include for instance, but are not restricted to, homoserine formation, pyroglutamic acid formation, disulphide bond formation, deamidation of asparagine or glutamine residues, methylation, t-butylation, t-butyloxycarbonylation, 4-methylbenzylation, thioanysilation, thiocresylation, benzyloxymethylation, 4-nitrophenylation, benzyloxycarbonylation, 2-nitrobencoylation, 2-nitrosulphenylation, 4-toluenesulphonylation, pentafluorophenylation, diphenylmethylation, 2-chlorobenzyloxycarbonylation, 2,4,5-trichlorophenylation, 2-bromobenzyloxycarbonylation, 9-fluorenylmethyloxycarbonylation, triphenylmethylation, 2,2,5,7,8,-pentamethylchroman-6-sulphonylation, hydroxylation, oxidation of methionine, formylation, acetylation, anisylation, benzylation, bencoylation, trifluoroacetylation, carboxylation of aspartic acid or glutamic acid, phosphorylation, sulphation, cysteinylation, glycolysation with pentoses, deoxyhexoses, hexosamines, hexoses or N-acetylhexosamines, farnesylation, myristolysation, biotinylation, palmitoylation, stearoylation, geranylgeranylation, glutathionylation, 5′-adenosylation, ADP-ribosylation, modification with N-glycolylneuraminic acid, N-acetylneuraminic acid, pyridoxal phosphate, lipoic acid, 4′-phosphopantetheine, or N-hydroxysuccinimide.

In the compounds of formula (3), the amino acid moieties A, B, C, D, and E are respectively attached to the adjacent moiety by amide bonds in a usual manner according to standard nomenclature so that the amino-terminus (N-terminus) of the amino acids (peptide) is drawn on the left and the carboxyl-terminus of the amino acids (peptide) is drawn on the right. (C-terminus).

Until the present invention by Applicants, known peptide substrates of the proline-specific serine protease dipeptidyl peptidase IV in vitro are the tripeptides Diprotin A (Ile-Pro-Ile), Diprotin B (Val-Pro-Leu) and Diprotin C (Val-Pro-Ile). Applicants have unexpectedly discovered that the compounds disclosed herein above and below act as substrates of dipeptidyl peptidase IV in vivo in a mammal and, in pharmacological doses, improve insulin sensitivity and islet signaling and alleviate pathological abnormalities of the metabolism of mammals such as glucosuria, hyperlipidaemia, metabolic acidosis and diabetes mellitus by competitive catalysis.

Preferred peptide compounds are listed in table 5.

TABLE 5
Examples of peptide substrates

| Peptide | Mass (calc.) | Mass (exp.)¹ [M + H⁺] |
|---|---|---|
| 2-Amino octanoic acid-Pro-Ile | 369.5 | 370.2 |
| Abu-Pro-Ile | 313.4 | 314.0 |
| Aib-Pro-Ile | 313.4 | 314.0 |
| Aze-Pro-Ile | 311.4 | 312.4 |
| Cha-Pro-Ile | 381.52 | 382.0 |
| Ile-Hyp-Ile | 356.45 | 358.2 |
| Ile-Pro-allo-Ile | 341.4 | 342.0 |
| Ile-Pro-t-butyl-Gly | 341.47 | 342.36 |
| Ile-Pro-Val | 327.43 | 328.5 |
| Nle-Pro-Ile | 341.45 | 342.2 |
| Nva-Pro-Ile | 327.43 | 328.2 |
| Orn-Pro-Ile | 342.42 | 343.1 |
| Phe-Pro-Ile | 375.47 | 376.2 |
| Phg-Pro-Ile | 361.44 | 362.2 |
| Pip-Pro-Ile | 338.56 | 340.0 |
| Ser(Bzl)-Pro-Ile | 405.49 | 406.0 |
| Ser(P)-Pro-Ile | 395.37 | 396.0 |
| Ser-Pro-Ile | 315.37 | 316.3 |
| t-butyl-Gly-Pro-D-Val | 327.4 | 328.6 |
| t-butyl-Gly-Pro-Gly | 285.4 | 286.3 |
| t-butyl-Gly-Pro-Ile | 341.47 | 342.1 |
| t-butyl-Gly-Pro-Ile-amide | 340.47 | 341.3 |
| t-butyl-Gly-Pro-t-butyl-Gly | 341.24 | 342.5 |
| t-butyl-Gly-Pro-Val | 327.4 | 328.4 |
| Thr-Pro-Ile | 329.4 | 330.0 |
| Tic-Pro-Ile | 387.46 | 388.0 |
| Trp-Pro-Ile | 414.51 | 415.2 |
| Tyr(P)-Pro-Ile | 471.47 | 472.3 |
| Tyr-Pro-allo-Ile | 391.5 | 392.0 |
| Val-Pro-allo-Ile | 327.4 | 328.5 |
| Val-Pro-t-butyl-Gly | 327.4 | 328.15 |
| Val-Pro-Val | 313.4 | 314.0 |

¹[M+H⁺] were determined by Electrospray mass spectrometry in positive ionization mode.

t-butyl-Gly is defined as:

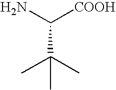

Ser(Bzl) and Ser(P) are defined as benzyl-serine and phosphoryl-serine, respectively. Tyr(P) is defined as phosphoryl-tyrosine.

Further preferred compounds, which can be used according to the present invention in combination with agents binding to the secondary binding site(s) of DP IV or DP IV-like enzymes, are peptidylketones of formula 4:

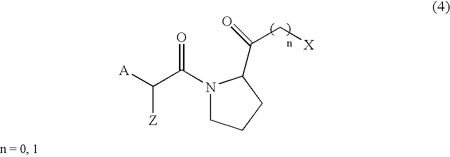

and pharmaceutically acceptable salts thereof, wherein:

A is selected from the following structures:

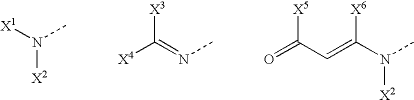

wherein

X¹ is H or an acyl or oxycarbonyl group including an amino acid residue, N-protected amino acid residue, a peptide residue or a N-protected peptide residue,

X² is H, —(CH)ₘ—NH—C₅H₃N—Y with m=2-4 or —C₅H₃N—Y (a divalent pyridyl residue) and Y is selected from H, Br, Cl, I, NO₂ or CN,

X³ is H or selected from an alkyl-, alkoxy-, halogen-, nitro-, cyano- or carboxy-substituted phenyl or from an alkyl-, alkoxy-, halogen-, nitro-, cyano- or carboxy-substituted pyridyl residue,

X⁴ is H or selected from an alkyl-, alkoxy-, halogen-, nitro-, cyano- or carboxy-substituted phenyl or from an alkyl-, alkoxy-, halogen-, nitro-, cyano- or carboxy-substituted pyridyl residue,

X⁵ is H or an alkyl, alkoxy or phenyl residue,

X⁶ is H or an alkyl residue,

for n=1

X is selected from: H, OR², SR², NR²R³, N⁺R²R³R⁴, wherein:

R² stands for acyl residues, which are optionally substituted with alkyl, cycloalkyl, aryl or heteroaryl residues, or for amino acid residues or peptidic residues, or alkyl residues, which are optionally substituted with alkyl, cycloalkyl, aryl or heteroaryl residues,

R³ stands for alkyl or acyl residues, wherein R² and R³ may be part of a saturated or unsaturated carbocyclic or heterocyclic ring,

R⁴ stands for alkyl residues, wherein R² and R⁴ or R³ and R⁴ may be part of a saturated or unsaturated carbocyclic or heterocyclic ring,

for n=0

X is selected from:

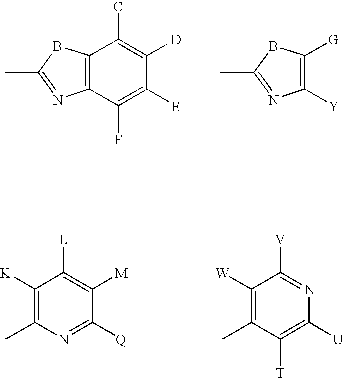

wherein

B stands for: O, S or NR⁵, wherein R⁵ is H, alkyl or acyl,

C, D, E, F, G, Y, K, L, M, Q, T, U, V and W are independently selected from alkyl and substituted alkyl residues, oxyalkyl, thioalkyl, aminoalkyl, carbonylalkyl, acyl, carbamoyl, aryl and heteroaryl residues, and

Z is selected from H, or a branched or straight chain alkyl residue from C₁-C₉, a branched or straight chain alkenyl residue from C₂-C₉, a cycloalkyl residue from C₃-C₈, a cycloalkenyl residue from C₅-C₇, an aryl or heteroaryl residue, or a side chain selected from all side chains of all natural amino acids or derivatives thereof.

In preferred compounds of formula 4, A is

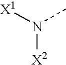

wherein

X¹ is H or an acyl or oxycarbonyl group including an amino acid residue, N-acylated amino acid residue, a peptide residue from di- to pentapeptides, preferably a dipeptide residue, or a N-protected peptide residue from di- to pentapeptides, preferably a N-protected dipeptide residue

X² is H, —(CH)ₘ—NH—C₅H₃N—Y with m=2-4 or —C₅H₃N—Y (a divalent pyridyl residue) and

Y is selected from H, Br, Cl, I, NO₂ or CN,

for n=1

X is preferably selected from: H, OR², SR², NR²R³, wherein:

R² stands for acyl residues, which are optionally substituted with alkyl, cycloalkyl, aryl or heteroaryl residues, or for amino acid residues or peptidic residues, or alkyl residues, which are optionally substituted with alkyl, cycloalkyl, aryl or heteroaryl residues,

R³ stands for alkyl or acyl residues, wherein R² and R³ may be part of a saturated or unsaturated carbocyclic or heterocyclic ring,

for n=0

X is preferably selected from:

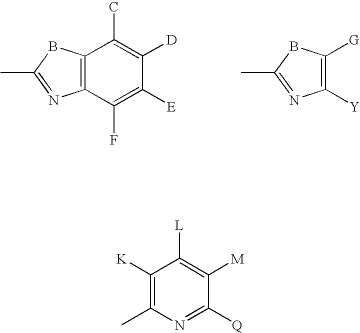

wherein

B stands for: O, S or NR⁵, wherein R⁵ is H, alkyl or acyl,

C, D, E, F, G, Y, K, L, M and Q are independently selected from alkyl and substituted alkyl residues, oxyalkyl, thioalkyl, aminoalkyl, carbonylalkyl, acyl, carbamoyl, aryl and heteroaryl residues, and

Z is selected from H, or a branched or straight chain alkyl residue from C₁-C₉, preferably C₂-C₆, a branched or straight chain alkenyl residue from C₂-C₉, a cycloalkyl residue from C₃-C₈, a cycloalkenyl residue from C₅-C₇, an aryl or heteroaryl residue, or a side chain selected from all side chains of all natural amino acids or derivatives thereof.

In more preferred compounds of formula 4, A is

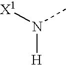

wherein

X¹ is H or an acyl or oxycarbonyl group including an amino acid residue, N-acylated amino acid residue or a peptide residue from di- to pentapeptides, preferably a dipeptide residue, or a N-protected peptide residue from di- to pentapeptides, preferably a N-protected dipeptide residue

for n=1,

X is preferably selected from: H, OR², SR², wherein:

R² stands for acyl residues, which are optionally substituted with alkyl or aryl residues,

for n=0

X is preferably selected from:

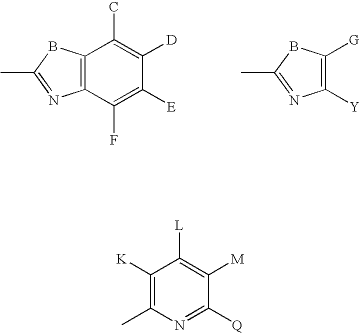

wherein

B stands for: O, S or NR⁵, wherein R⁵ is H, alkyl or acyl,

C, D, E, F, G, Y, K, L, M, Q, T, U, V and W are independently selected from alkyl and substituted alkyl residues, oxyalkyl, thioalkyl, aminoalkyl, carbonylalkyl, acyl, carbamoyl, aryl and heteroaryl residues, and

Z is selected from H, or a branched or straight chain alkyl residue from C₁-C₉, preferably C₂-C₆, a branched or straight chain alkenyl residue from C₂-C₉, a cycloalkyl residue from C₃-C₈, a cycloalkenyl residue from C₅-C₇, an aryl or heteroaryl residue, or a side chain selected from all side chains of all natural amino acids or derivatives thereof.

In most preferred compounds of formula 4, A is

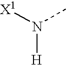

wherein

X¹ is H or an acyl or oxycarbonyl group including an amino acid residue, N-acylated amino acid residue or a dipeptide residue, containing a Pro or Ala in the penultimate position, or a N-protected dipeptide residue containing a Pro or Ala in the penultimate position,

for n 1,

X is H,

for n=0

X is preferably selected from:

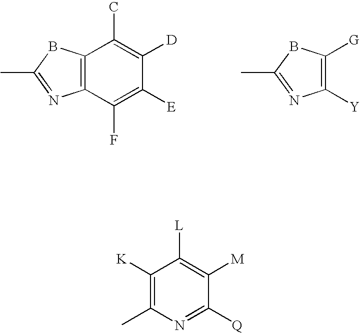

wherein

B stands for: O or S, most preferably for S

C, D, E, F, G, Y, K, L, M, Q, are H and

Z is selected from H, or a branched or straight chain alkyl residue from C₃-C₅, a branched or straight chain alkenyl residue from C₂-C₉, a cycloalkyl residue from C₅-C₇, a cycloalkenyl residue from C₅-C₇, an aryl or heteroaryl residue, or a side chain selected from all side chains of all natural amino acids or derivatives thereof.

Most preferred for Z is H.

According to a preferred embodiment the acyl groups are C₁-C₆-acyl groups.

According to another further preferred embodiment the alk(yl) groups are C₁-C₆-alk(yl) groups, which may be branched or unbranched.

According to a still further preferred embodiment the alkoxy groups are C₁-C₆-alkoxy groups.

According to yet another further preferred embodiment the aryl residues are C₅-C₁₂ aryl residues that have optionally fused rings.

According to a still yet further preferred embodiment the cycloalkyl residues (carbocycles) are C₃-C₈-cycloalkyl residues.

According to a still further preferred embodiment the heteroaryl residues are C₄-C₁₁ aryl residues that have optionally fused rings and, in at least one ring, additionally from 1 to 4 preferably 1 or 2 hetero atoms, such as O, N and/or S.

According to a still yet further preferred embodiment peptide residues are corresponding residues containing from 2 to 50 amino acids.

According to a further preferred embodiment the heterocyclic residues are C₂-C₇-cycloalkyl radicals that additionally have from 1 to 4, preferably 1 or 2 hetero atoms, such as O, N and/or S.

According to another still further preferred embodiment the carboxy groups are C₁-C₆ carboxy groups, which may be branched or unbranched.

According to yet another still further preferred embodiment the oxycarbonyl groups are groups of the formula —O—(CH₂)₁₋₆COOH.

The amino acids can be any natural or synthetic amino acid, preferably natural alpha amino acids. Further, according to the present invention compounds of formulas 5, 6, 7, 8, 9, 10 and 11, including all stereoisomers and pharmaceutical acceptable salts thereof can be used in combination with agents binding to the secondary binding site(s) of DP IV or DP IV-like enzymes:

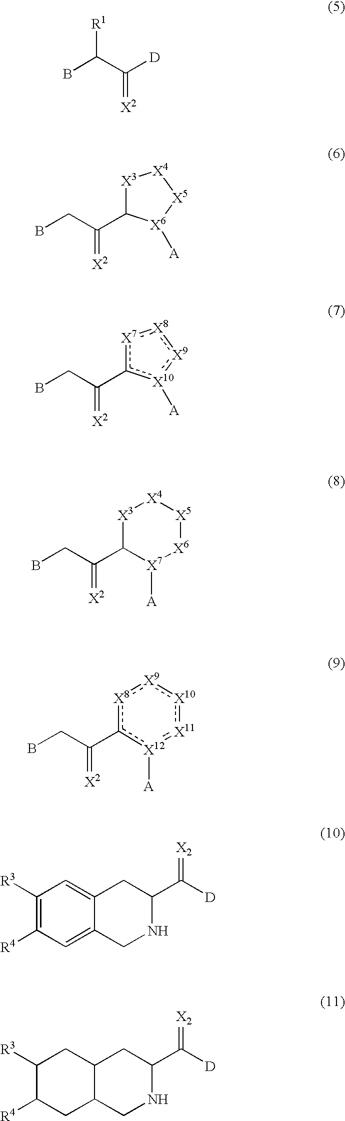

wherein:

R¹ is H, a branched or linear C₁-C₉ alkyl residue, a branched or linear C₂-C₉ alkenyl residue, a C₃-C₈ cycloalkyl-, C₅-C₇ cycloalkenyl-, aryl- or heteroaryl residue or a side chain of a natural amino acid or a derivative thereof,

R³ and R⁴ are selected from H, hydroxy, alkyl, alkoxy, aryloxy, nitro, cyano or halogen,

A is H or an isoster of a carbonic acid, like a functional group selected from CN, SO₃H, CONHOH, PO₃R⁵R⁶, tetrazole, amide, ester, anhydride, thiazole and imidazole,

B is selected from:

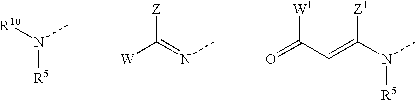

wherein

R⁵ is H, —(CH)ₙ—NH—C₅H₃N—Y with n=2-4 and C₅H₃N—Y (a divalent pyridyl residue) with Y═H, Br, Cl, I, NO₂ or CN,

R¹⁰ is H, an acyl, oxycarbonyl or a amino acid residue,

W is H or a phenyl or pyridyl residue, unsubstituted or substituted with one, two or more alkyl, alkoxy, halogen, nitro, cyano or carboxy residues,

W¹ is H, an alkyl, alkoxy or phenyl residue,

Z is H or a phenyl or pyridyl residue, unsubstituted or substituted with one, two or more alkyl, alkoxy, halogen, nitro, cyano or carboxy residues,

Z¹ is H or an alkyl residue,

D is a cyclic C₄-C₇ alkyl, C₄-C₇ alkenyl residue which can be unsubstituted or substituted with one, two or more alkyl groups or a cyclic 4-7-membered heteroalkyl or a cyclic 4-7-membered heteroalkenyl residue,

X² is O, NR⁶, N⁺(R⁷)₂, or S,

X³ to X¹² are independently selected from CH₂, CR⁸R⁹, NR⁶, N⁺(R⁷)₂, O, S, SO and SO₂, including all saturated and unsaturated structures,

R⁶, R⁷, R⁸, R⁹ are independently selected from H, a branched or linear C₁-C₉ alkyl residue, a branched or linear C₂-C₉ alkenyl residue, a C₃-C₈ cycloalkyl residue, a C₅-C₇ cycloalkenyl residue, an aryl or heteroaryl residue,

with the following provisos:

Formula 6: X⁶ is CH if A is not H,

Formula 7: X¹⁰ is C if A is not H,

Formula 8: X⁷ is CH if A is not H,

Formula 9: X¹² is C if A is not H.

Throughout the description and the claims the expression “acyl” can denote a C1-20 acyl residue, preferably a C1-8 acyl residue and especially preferred a C1-4 acyl residue; “cycloalkyl” can denote a C3-12 cycloalkyl residue, preferably a C4, C5 or C6 cycloalkyl residue; and “carbocyclic” can denote a C3-12 carbocyclic residue, preferably a C4, C5 or C6 carbocyclic residue. “Heteroaryl” is defined as an aryl residue, wherein 1 to 4, and more preferably 1, 2 or 3 ring atoms are replaced by heteroatoms like N, S or O. “Heterocyclic” is defined as a cycloalkyl residue, wherein 1, 2 or 3 ring atoms are replaced by heteroatoms like N, S or O. “Peptides” are selected from dipeptides to decapeptides, preferred are dipeptides, tripeptides, tetrapeptides and pentapeptides. The amino acids for the formation of the “peptides” can be selected from those listed above.

Because of the wide distribution of the protein in the body and the wide variety of mechanisms involving DP IV, DP IV-activity and DP IV-related proteins, systemic therapy (enteral or parenteral administration) with DP IV-inhibitors can result in a series of undesirable side-effects.

The problem to be solved was moreover, to provide compounds that can be used, in combination with agents binding to the secondary binding site(s) of DP IV or DP IV-like enzymes, for targeted influencing of locally limited patho-physiological and physiological processes. The problem of the invention especially consists in obtaining locally limited and highly specific inhibition of DP IV or DP IV-analogous activity for the purpose of targeted intervention in the regulation of the activity of locally active substrates.

This problem is solved according to the invention by the use compounds of the general formula (12) in combination with agents binding to the secondary binding site(s) of DP IV or DP IV-like enzymes:

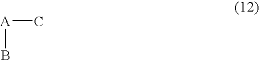

wherein

A is an amino acid having at least one functional group in the side chain,

B is a chemical compound covalently bound to at least one functional group of the side chain of A,

C is a thiazolidine, pyrrolidine, cyanopyrrolidine, hydroxyproline, dehydroproline or piperidine group amide-bonded to A.

In accordance with a preferred embodiment of the invention, pharmaceutical compositions are used comprising at least one compound of the general formula (12) and at least one customary adjuvant appropriate for the site of action.

Preferably A is an α-amino acid, especially a natural α-amino acid having one, two or more functional groups in the side chain, preferably threonine, tyrosine, serine, arginine, lysine, aspartic acid, glutamic acid or cysteine.

Preferably B is an oligopeptide having a chain length of up to 20 amino acids, a polyethylene glycol having a molar mass of up to 20 000 g/mol, an optionally substituted organic amine, amide, alcohol, acid or aromatic compound having from 8 to 50 C atoms.

Throughout the description and the claims the expression “alkyl” can denote a C₁₋₅₀ alkyl group, preferably a C₆₋₃₀ alkyl group, especially a C₈₋₁₂ alkyl group; for example, an alkyl group may be a methyl, ethyl, propyl, isopropyl or butyl group. The expression “alk”, for example in the expression “alkoxy”, and the expression “alkan”, for example in the expression “alkanoyl”, are defined as for “alkyl”; aromatic compounds are preferably substituted or optionally unsubstituted phenyl, benzyl, naphthyl, biphenyl or anthracene groups, which preferably have at least 8 C atoms; the expression “alkenyl” can denote a C₂₋₁₀ alkenyl group, preferably a C₂₋₆ alkenyl group, which has the double bond(s) at any desired location and may be substituted or unsubstituted; the expression “alkynyl” can denote a C₂₋₁₀ alkynyl group, preferably a C₂₋₆ alkynyl group, which has the triple bond(s) at any desired location and may be substituted or unsubstituted; the expression “substituted” or substituent can denote any desired substitution by one or more, preferably one or two, alkyl, alkenyl, alkynyl, mono- or multi-valent acyl, alkanoyl, alkoxyalkanoyl or alkoxyalkyl groups; the afore-mentioned substituents may in turn have one or more (but preferably zero) alkyl, alkenyl, alkynyl, mono- or multi-valent acyl, alkanoyl, alkoxyalkanoyl or alkoxyalkyl groups as side groups; organic amines, amides, alcohols or acids, each having from 8 to 50 C atoms, preferably from 10 to 20 C atoms, can have the formulae (alkyl)₂N— or alkyl-NH—, —CO— N(alkyl)₂ or —CO—NH(alkyl), -alkyl-OH or -alkyl-COOH.

Despite an extended side chain function, the compounds of formula (12) can still bind to the active centre of the enzyme dipeptidyl peptidase IV and analogous enzymes but are no longer actively transported by the peptide transporter PepT1. The resulting reduced or greatly restricted transportability of the compounds according to the invention leads to local or site directed inhibition of DP IV and DP IV-like enzyme activity.

By extending/expanding the side chain modifications, for example beyond a number of seven carbon atoms, it is accordingly possible to obtain a dramatic reduction in transportability. With increasing spatial size of the side chains, there is a reduction in the transportability of the substances. By spatially and sterically expanding the side chains, for example beyond the atom group size of a monosubstituted phenyl radical, hydroxylamine radical or amino acid residue, it is possible according to the invention to modify or suppress the transportability of the target substances.

Preferred compounds of formula (12) are compounds, wherein the oligopeptides have chain lengths of from 3 to 15, especially from 4 to 10, amino acids, and/or the polyethylene glycols have molar masses of at least 250 g/mol, preferably of at least 1500 g/mol and up to 15 000 g/mol, and/or the optionally substituted organic amines, amides, alcohols, acids or aromatic compounds have at least 12 C atoms and preferably up to 30 C atoms.

The compounds of the present invention can be converted into and used as acid addition salts, especially pharmaceutically acceptable acid addition salts. The pharmaceutically acceptable salt generally takes a form in which an amino acids basic side chain is protonated with an inorganic or organic acid. Representative organic or inorganic acids include hydrochloric, hydrobromic, perchloric, sulfuric, nitric, phosphoric, acetic, propionic, glycolic, lactic, succinic, maleic, fumaric, malic, tartaric, citric, benzoic, mandelic, methanesulfonic, hydroxyethanesulfonic, benzenesulfonic, oxalic, pamoic, 2-naphthalenesulfonic, p-toulenesulfonic, cyclohexanesulfamic, salicylic, saccharinic or trifluoroacetic acid. All pharmaceutically acceptable acid addition salt forms of the compounds of formulas (1) to (12) are intended to be embraced by the scope of this invention.

In view of the close relationship between the free compounds and the compounds in the form of their salts, whenever a compound is referred to in this context, a corresponding salt is also intended, provided such is possible or appropriate under the circumstances.

The present invention further includes within its scope prodrugs of the compounds of formulas (1) to (12). In general, such prodrugs will be functional derivatives of the compounds which are readily convertible in vivo into the desired therapeutically active compound. Thus, in these cases, the present invention shall encompass the treatment of the various disorders described with prodrug versions of one or more of the claimed compounds, which convert to the above specified compound in vivo after administration to the subject. Conventional procedures for the selection and preparation of suitable prodrug derivatives are described, for example, in “Design of Prodrugs”, ed. H. Bundgaard, Elsevier, 1985 and the patent applications DE 198 28 113 and DE 198 28 114, which are fully incorporated herein by reference.

Where the compounds or prodrugs according to this invention have at least one chiral center, they may accordingly exist as enantiomers. Where the compounds or prodrugs possess two or more chiral centers, they may additionally exist as diastereomers. It is to be understood that all such isomers and mixtures thereof are encompassed within the scope of the present invention. Furthermore, some of the crystalline forms of the compounds or prodrugs may exist as polymorphs and as such are intended to be included in the present invention. In addition, some of the compounds may form solvates with water (i.e. hydrates) or common organic solvents, and such solvates are also intended to be encompassed within the scope of this invention.

The compounds, including their salts, can also be obtained in the form of their hydrates, or include other solvents used for their crystallization.

In a further illustrative embodiment, the present invention provides formulations for agents binding to the secondary binding site of DP IV or DP IV-like enzymes allone or in combination with DP IV-inhibitors, e.g. the compounds of formulas (1) to (12), and their corresponding pharmaceutically acceptable prodrugs and acid addition salt forms, in pharmaceutical compositions.

The term “subject” as used herein, refers to an animal, preferably a mammal, most preferably a human, who has been the object of treatment, observation or experiment.

The term “therapeutically effective amount” as used herein, means that amount of active compound or pharmaceutical agent that elicits the biological or medicinal response in a tissue system, animal or human, being sought by a researcher, veterinarian, medical doctor or other clinician, which includes alleviation of the symptoms of the disease or disorder being treated.

To prepare the pharmaceutical compositions of this invention, one or more compounds capable of binding to the secondary binding site and/or DP IV-inhibitors or salts thereof of the invention can be used as the active ingredient(s). The active ingredient(s) is intimately admixed with a pharmaceutical carrier according to conventional pharmaceutical compounding techniques, which carrier may take a wide variety of forms depending of the form of preparation desired for administration, e.g., oral or parenteral such as intramuscular. In preparing the compositions in oral dosage form, any of the usual pharmaceutical media may be employed. Thus, for liquid oral preparations, such as for example, suspensions, elixirs and solutions, suitable carriers and additives include water, glycols, oils, alcohols, flavoring agents, preservatives, coloring agents and the like; for solid oral preparations such as, for example, powders, capsules, gelcaps and tablets, suitable carriers and additives include starches, sugars, diluents, granulating agents, lubricants, binders, disintegrating agents and the like. Because of their ease in administration, tablets and capsules represent the most advantageous oral dosage unit form, in which case solid pharmaceutical carriers are obviously employed. If desired, tablets may be sugar coated or enteric coated by standard techniques. For parenterals, the carrier will usually comprise sterile water, through other ingredients, for example, for purposes such as aiding solubility or for preservation, may be included.

Injectable suspensions may also prepared, in which case appropriate liquid carriers, suspending agents and the like may be employed. The pharmaceutical compositions herein will contain, per dosage unit, e.g., tablet, capsule, powder, injection, teaspoonful and the like, an amount of the active ingredient(s) necessary to deliver an effective dose as described above. The pharmaceutical compositions herein will contain, per dosage unit, e.g., tablet, capsule, powder, injection, suppository, teaspoonful and the like, from about 0.03 mg to 100 mg/kg (preferred 0.1-30 mg/kg) and may be given at a dosage of from about 0.1-300 mg/kg/day (preferred 1-50 mg/kg/day) of each active ingredient or combination thereof. The dosages, however, may be varied depending upon the requirement of the patients, the severity of the condition being treated and the compound being employed. The use of either daily administration or post-periodic dosing may be employed.

Preferably these compositions are in unit dosage forms from such as tablets, pills, capsules, powders, granules, sterile parenteral solutions or suspensions, metered aerosol or liquid sprays, drops, ampoules, autoinjector devices or suppositories; for oral parenteral, intranasal, sublingual or rectal administration, or for administration by inhalation or insufflation. Alternatively, the composition may be presented in a form suitable for once-weekly or once-monthly administration; for example, an insoluble salt of the active compound, such as the decanoate salt, may be adapted to provide a depot preparation for intramuscular injection. For preparing solid compositions such as tablets, the principal active ingredient is mixed with a pharmaceutical carrier, e.g. conventional tableting ingredients such as corn starch, lactose, sucrose, sorbitol, talc, stearic acid, magnesium stearate, dicalcium phosphate or gums, and other pharmaceutical diluents, e.g. water, to form a solid preformulation composition containing a homogeneous mixture of a compound of the present invention, or a pharmaceutically acceptable salt thereof. When referring to these preformulation compositions as homogeneous, it is meant that the active ingredient is dispersed evenly throughout the composition so that the composition may be readily subdivided into equally effective dosage forms such as tablets, pills and capsules. This solid preformulation composition is then subdivided into unit dosage forms of the type described above containing from 0.1 to about 500 mg of each active ingredient or combinations thereof of the present invention.

The tablets or pills of the compositions of the present invention can be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer which serves to resist disintegration in the stomach and permits the inner component to pass intact into the duodenum or to be delayed in release. A variety of material can be used for such enteric layers or coatings, such materials including a number of polymeric acids with such materials as shellac, cetyl alcohol and cellulose acetate.

This liquid forms in which the compositions of the present invention may be incorporated for administration orally or by injection include, aqueous solutions, suitably flavoured syrups, aqueous or oil suspensions, and flavoured emulsions with edible oils such as cottonseed oil, sesame oil, coconut oil or peanut oil, as well as elixirs and similar pharmaceutical vehicles. Suitable dispersing or suspending agents for aqueous suspensions, include synthetic and natural gums such as tragacanth, acacia, alginate, dextran, sodium carboxymethylcellulose, methylcellulose, polyvinylpyrrolidone or gelatin.

Where the processes for the preparation of the compounds according to the invention give rise to mixture of stereoisomers, these isomers may be separated by conventional techniques such as preparative chromatography. The compounds may be prepared in racemic form, or individual enantiomers may be prepared either by enantiospecific synthesis or by resolution. The compounds may, for example, be resolved into their components enantiomers by standard techniques, such as the formation of diastereomeric pairs by salt formation with an optically active acid, such as (−)-di-p-toluoyl-d-tartaric acid and/or (+)-di-p-toluoyl-1-tartaric acid followed by fractional crystallization and regeneration of the free base. The compounds may also resolved by formation of diastereomeric esters or amides, followed by chromatographic separation and removal of the chiral auxiliary. Alternatively, the compounds may be resolved using a chiral HPLC column.

During any of the processes for preparation of the compounds of the present invention, it may be necessary and/or desirable to protect sensitive or reactive groups on any of the molecules concerned. This may be achieved by means of conventional protecting groups, such as those described in Protective Groups in Organic Chemistry, ed. J. F. W. McOmie, Plenum Press, 1973; and T. W. Greene & P. G. M. Wuts, Protective Groups in Organic Synthesis, John Wiley & Sons, 1991. The protecting groups may be removed at a convenient subsequent stage using conventional methods known from the art.

The method of treating conditions modulated by the dipeptidyl peptidase IV or dipeptidyl peptidase IV-like enzymes described in the present invention may also be carried out using a pharmaceutical composition comprising any or any combination of the compounds as defined herein and a pharmaceutically acceptable carrier. The pharmaceutical composition may contain between about 0.01 mg and 100 mg, preferably about 5 to 50 mg, of each compound, and may be constituted into any form suitable for the mode of administration selected. Carriers include necessary and inert pharmaceutical excipients, including, but not limited to, binders, suspending agents, lubricants, flavorants, sweeteners, preservatives, dyes, and coatings. Compositions suitable for oral administration include solid forms, such as pills, tablets, caplets, capsules (each including immediate release, timed release and sustained release formulations), granules, and powders, and liquid forms, such as solutions, syrups, elixirs, emulsions, and suspensions. Forms useful for parenteral administration include sterile solutions, emulsions and suspensions.

Advantageously, compounds of the present invention may be administered in a single daily dose, or the total daily dosage may be administered in divided doses of two, three or four times daily. Furthermore, compounds for the present invention can be administered in intranasal form via topical use of suitable intranasal vehicles, or via transdermal skin patches well known to those of ordinary skill in that art. To be administered in the form of transdermal delivery system, the dosage administration will, of course, be continuous rather than intermittent throughout the dosage regimen.

For instance, for oral administration in the form of a tablet or capsule, the active drug component can be combined with an oral, non-toxic pharmaceutically acceptable inert carrier such as ethanol, glycerol, water and the like. Moreover, when desired or necessary, suitable binders; lubricants, disintegrating agents and coloring agents can also be incorporated into the mixture. Suitable binders include, without limitation, starch, gelatin, natural sugars such as glucose or betalactose, corn sweeteners, natural and synthetic gums such as acacia, tragacanth or sodium oleate, sodium stearate, magnesium stearate, sodium benzoate, sodium acetate, sodium chloride and the like. Disintegrators include, without limitation, starch, methyl cellulose, agar, bentonite, xanthan gum and the like.

The liquid forms in suitable flavored suspending or dispersing agents such as the synthetic and natural gums, for example, tragacanth, acacia, methyl-cellulose and the like. For parenteral administration, sterile suspensions and solutions are desired. Isotonic preparations which generally contain suitable preservatives are employed when intravenous administration is desired.

The compounds of the present invention can also be administered in the form of liposome delivery systems, such as small unilamellar vesicles, large unilamellar vesicles, and multilamellar vesicles. Liposomes can be formed from a variety of phospholipids, such as cholesterol, stearylamine or phosphatidylcholines.

Compounds of the present invention may also be delivered by the use of monoclonal antibodies as individual carriers to which the compound molecules are coupled. The compounds of the present invention may also be coupled with soluble polymers as targetable drug carriers. Such polymers can include polyvinylpyrrolidone, pyran copolymer, polyhydroxypropylmethacrylamidephenol, polyhydroxyethylaspartamid-ephenol, or polyethyl eneoxidepolyllysine substituted with palmitoyl residue. Furthermore, the compounds of the present invention may be coupled to a class of biodegradable polymers useful in achieving controlled release of a drug, for example, polyactic acid, polyepsilon caprolactone, polyhydroxy butyeric acid, polyorthoesters, polyacetals, polydihydropyrans, polycyanoacrylates and cross-linked or amphipathic block copolymers of hydrogels.

Compounds of this invention may be administered in any of the foregoing compositions and according to dosage regimens established in the art whenever treatment of the addressed disorders is required.

The daily dosage of the products may be varied over a wide range from 0.01 to 1.000 mg per mammal per day. For oral administration, the compositions are preferably provided in the form of tablets containing, 0.01, 0.05, 0.1, 0.5, 1.0, 2.5, 5.0, 10.0, 15.0, 25.0, 50.0, 100, 150, 200, 250 and 500 milligrams of each active ingredient or combinations thereof for the symptomatic adjustment of the dosage to the patient to be treated. An effective amount of the drug is ordinarily supplied at a dosage level of from about 0.1 mg/kg to about 300 mg/kg of body weight per day. Preferably, the range is from about 1 to about 50 mg/kg of body weight per day. The compounds may be administered on a regimen of 1 to 4 times per day.

Optimal dosages to be administered may be readily determined by those skilled in the art, and will vary with the particular compound used, the mode of administration, the strength of the preparation, the mode of administration, and the advancement of disease condition. In addition, factors associated with the particular patient being treated, including patient age, weight, diet and time of administration, will result in the need to adjust dosages.

### EXAMPLES

### Example 1

### Determination of the Half-Life (t_{1/2})

Matrix-assisted laser-desorption ionization time of flight mass spectrometry (MALDI-TOF MS) experiments were carried out at 30° C. at pH 7.6 in 0.1 M Tris/HCl (Sigma-Aldrich, Deisenhofen, Germany) buffer with 25 μM peptide solution. The degradation fate of peptides was measured by monitoring the signal intensity of the pseudomolecular ion peaks of parent peptides and N-terminal shorted peptides versus time when incubated with 40 mU procine DP IV, recombinant human DP IV or serum DP IV activity. The enzyme was preincubated with hexapeptide TFTSDY (15 min, 30° C., 0.016M, 1:1 with DP IV, the TFTSDY concentration in the reaction mixture was 160 μM). As control served the preincubation of DP IV with 0.01M Tris-buffer (Sigma-Aldrich, Deisenhofen, Germany). The mass spectrometer employed was a Hewlett-Packard G2025 model with a linear time of flight analyzer; samples (4 μL) were mixed 1:1 v/v with matrix (44 mg diammonium-hydrogen-citrate and 30 mg 2′,6′-dihydroxyacetophenone in 1 ml aqueous solution containing 50% acetonitrile and 0.05% trifluoroacetic acid; Sigma-Aldrich), transferred to a probe tip and immediately evaporated using the Hewlett-Packard G2024A (Hewlett-Packard, Waldbronn, Germany) sample preparation vacuum chamber. 250 single laser-shot spectra were accumulated. This method of monitoring biodegradation has been validated and allows the general comparison of half-degradation times (t_{1/2}) under various conditions.

The t_{1/2}-calculation followed this procedure:

The height of the substrate peak was measured and set as 100% at time=0. During the reaction course the sum of substrate and product peak height were set as 100% and the percentage of the remaining substrate peak (also expressed as relative concentration) was determined. Diagrammed relative substrate concentration versus time t_{1/2} can be calculated based on first order exponential decay reaction course.

$$\begin{matrix}A {⟶}^{{k}_{1}} B \\ v = - \frac{ⅆ [ A ]}{ⅆ [ t ]} = {k}_{1} * [ A ] \\ - {\int }_{{A}_{0}}^{{A}_{1}} \frac{ⅆ [ A ]}{[ A ]} = {\int }_{{t}_{0}}^{t} {k}_{1} ⅆ t \\ [ A ] = {[ A ]}_{0} {ⅇ}^{- {k}_{1} t} \\ {k}_{1} = \frac{ln 2}{{t}_{1 / 2}}\end{matrix}$$

Legend:

A substrate (bioactive peptide)

B product (N-terminal truncated bioactive peptide)

Kᵢ first order rate constant

Kₘ Michaelis-Menten-constant

v₁ initial rate of the reaction

Vₘₐₓ maximal rate of the reaction

[S] substrate concentration

### Example 2

### Determination of Kᵢ:

In order to measure the inhibition constant Kᵢ a photometric assay was used The peptides were measured as competitors of the standard substrate GP-4-Nitroanilide. Three different substrate concentrations (0.4 mM to 0.05 mM) were combined with 8 different competitor concentrations (0.5 mM to 2 μM). The reaction was started by addition of 3.5 nM DP IV. Experiments were carried out under standard conditions: 30° C. in pH 7.6 40 mM HEPES (Sigma-Aldrich) buffer. Nitroaniline production was monitored using a HTS 7000+ microplate reader (PerkinElmer, Überlingen, Germany). The K₁-values were calculated via non-linear regression using the enzyme kinetic program Grafit 4.016 (Erithacus Ltd, UK).

For a reversible competitive inhibition is to assumed:

$${v}_{i} = \frac{{V}_{max} * {K}_{m}}{[ S ] + {K}_{m} ( 1 + \frac{[ I ]}{{K}_{i}} )}$$

Legend.

[I] inhibitor concentration

Kᵢ inhibition constant

### Example 3

### MALDI-TOF Approach

In order to investigate directly the influence of TFTSDY on the DP IV-catalyzed peptide hydrolysis the MALDI-TOF assay was used.

As described before (determination of t_{1/2}) DP IV and TFTSDY were preincubated and the reaction was started by adding the enzyme/hexapeptide mixture to substrate/buffer mix. The control reaction mixture consisted of buffer, enzyme and substrate. From the curves of the first order exponential the initial rate (v₁) for the control and the reversible inhibited reaction was calculated.

For the uninfluenced reaction the Michaelis-Menten-equation was used.

Vᵢ was calculated from plotting the relative substrate concentration versus time.

Kₘ is given, also the substrate concentration.

$${v}_{i} = \frac{{V}_{max} * {K}_{m}}{{K}_{m} + [ S ]}$$

For the reversible inhibited reaction the following reaction was used to calculate Kₗ:

$${v}_{i} = \frac{{V}_{max} * {K}_{m}}{[ S ] + {K}_{m} ( 1 + \frac{[ I ]}{{K}_{i}} )}$$

### Example 3

### Determination of Kₘ

Experiments were carried out with a capillary zone electrophoresis apparatus (MDQ, Beckmann, München, Germany).

The reaction mixture contained 50 μl Gly-Gly (100 mM as standard), 50 μM substrate solved in 0.01 M sodium phosphat buffer (pH 7.6) and 10 μl DP IV (40 mU/ml) stored at 30° C. Six substrate concentrations varying from 1 μM to 60 μM were measured. As running buffer 0.1 M sodium phosphat buffer, pH 2.5 was used. A sample from the reaction mixture was injected with 0.5 psi over 5 s at predefined time points. Separation was carried out in a capillary with 50 μM inner diameter and 20 cm effective length. The following separation parameters were used:

| | |
|---|---|
| Separation voltage: | 16 kV |
| Separation time: | 12 min |
| Separation temperature: | 25° C. |
| Detection wave length: | 200 nm |

The maximal rate was calculated by plotting product concentration versus time. The Kₘ-value was calculated transferring the data in the Michaelis-Menten-equation (GraFit 4.0.16, Erithacus Ltd., UK).

### Example 4

### Expression, Fermentation and Purification of Human DP IV and its Mutant Variants

Strains and Plasmid:

P. pastoris strain X-33 and the vector pPICαC were purchased from Invitrogen (USA). E. coli XL-10 cells were provided from Stratagene (USA).

Plasmid Construction and DNA Sequencing.

The DP IV encoding region (Δ1-36) plus his₆-tag contained in a pcDNA-3.1 vector was amplified using primers DP IV-21 (TCATCGATGCATCATCATCATCATCAT) and DP IV-22 (TAGGTACCGCTAAGGTAAAGAGAAAC) while implementing the restriction sites for KpnI and BspD1. This fragment was digested with the restriction enzymes KpnI and BspD1 as well as the vector pPCR-ScriptCam (Stratagene, USA), afterwards vector and PCR product were ligated and transformed into the E. coli-strain XL-10. Insertion and orientation was confirmed applying restriction enzyme analysis and partial sequencing. That was followed by excision of the DP IV encoding region from the pPCR-ScriptCam vector with the same restriction enzymes KpnI and BspD1 and its ligation into the Pichia vector pPICαC, which was also treated with the same restriction enzymes before.

Site Directed Mutagenesis:

Single amino acid mutations were carried out with the Quick Change Site-directed Mutagenesis Kit from Stratagene (USA). Following primers were used to introduce the mutations:

R310A-DP IV:

| | |
|---|---|
| DP IV-84 | GACATGGGCAACACAAGAAGCAATTTCTTTGCAGTGGC |
| DP IV-85 | GCCACTGCAAAGAAATTGCTTCTTGTGTTGCCCATGTC |

R560A-DP IV:

| | |
|---|---|
| DP IV-73: | GCAGACACTGTCTTCGCACTGAACTGGGCCACTTACC |
| DP IV-74b: | GGTAAGTGGCCCAGTTCAGTGCGAAGACAGTGTCTGC |

W629A-DP IV:

| | |
|---|---|
| DP IV-75: | GCAATTTGGGGCTGGTCATAGCGAGGGTACGTAACC |
| DP IV 76: | GGTTACGTACCCTCGCTATGACCAGCCCCAAATTGC. |

Transformation of P. pastoris X-33:

The vector pPICαC containing the DP IV-variants was linearized using the restriction enzyme Sac I. Transformation was carried out with an electroporation system from BioRad (Germany) according to the Invitrogen Pichia expression kit manual.

Media and Buffers:

YPD, BMMY, and BMGY for shake flask expression were prepared as described in the Invitrogen Pichia expression kit manual using reagents obtained from Difco. Media for fermentation were composed as described in the Invitrogen Pichia fermentation process guidelines using chemicals purchased from Sigma (Deisenhofen, Germany).

Small-Scale Expression Studies:

Single colonies were grown in BMGY at 250 rpm, 28° C. overnight. Induction of gene expression was initiated after a media exchange to BMMY. DP IV activity in the expression medium was assayed after 48 hours. Clones displaying highest activity were further monitored in a shaking flask culture (15 ml BMGY and 15 ml BMMY respectively) regarding growth rate and expression rate.

Fermentation:

The clone displaying the highest DP IV activity was used to inoculate 5 ml of BMGY. After 16-18 h of growth at 250 rpm and 28° C. 1 ml of the culture was used to start a 200 ml BMGY flask shake preculture. The cells were grown for 16-18 h at 28° C. A 21 fermentation was started with the 200 ml inoculum according to the Invitrogen Pichia fermentation process guidelines.

Purification of DP IV:

Expression medium was centrifuged at 40,000*g for 20 minutes to pellet the yeast cells. The supernatant was filtered to remove any residual solids using a 45 μM cellulose acetate filter from Satorius (Germany). Medium was adjusted to pH 7.6 while adding 300 mM NaCl and 50 mM sodium phosphate buffer.

Affinity chromatography was carried out at 4° C. with a Ni-NTA sepharose column (Qiagen, Germany). The column was pre-equilibrated with 300 mM NaCl, 50 mM NaH₂PO₄-buffer pH 7.6. The enzyme was eluted with 250 mM imidazole. DP IV assay and SDS-PAGE monitored the purification process. The fractions with the highest DP IV content was further concentrated by ultra-filtration in an Amicon apparatus (cut off 10 kDa) to 0.5 ml.

Gel Filtration:

The 0.5 ml ultra-filtrate were applied to a Superdex 200 HiLoad 26/60 column (Pharmacia, Upsalla, Sweden) with a flow rate at 0.25 ml/min using a 300 mM NaCl, 50 mM NaH₂PO₄-buffer pH 7.6 at 4° C. The purification process was monitored via SDS-PAGE and activity assay.

DP IV Assay:

DP IV activity assays were performed spectrofluorimetrically using H-Gly-Pro-AMC (Bachem, Heidelberg, Germany) as substrate and a 0.1M HEPES buffer pH 7.6 plus 0.05M NaCl (Sigma, Deisenhofen, Germany) while monitoring the releasing of AMC by DP IV (λ_{excitation}=380 nm; λₑₘᵢₛₛᵢₒₙ=460 nm).

SDS-PAGE Analysis:

Proteins were analysed by SDS-PAGE using 12% separating gels with 3% stacking gel. Gels were stained applying Coomassie brilliant blue R-250.

Protein Determination:

Protein concentrations were determined using the BioRad (Germany) Bradford assay kit according to the instructions of the manufacturer.

Western Blot Analysis:

Analytical gel electrophoreses in SDS-polyacrylamid gels were performed according to Laemmli [1] with separation gels containing 12% acrylamide. The seperated proteins were transferred to a nitrocellulose membrane (Schleicher&Schuell, Germany) following standard procedures. To detect his-tagged protein a penta-his-tag-antibody and a secondary antibody provided from Qiagen (Germany) (1:2000) was used. Chemo-luminescence was assayed according to the manufacturers protocol (SuperSignal™ West Pico, PIERCE).

Substrates:

All investigated bioactive peptides were obtained from Bachem (Heidelberg, Germany), with exception of glucagon, GIP and its analogs and fragments. These peptides were synthesized at applicant's laboratories.

### Example 5

### Synthesis of DP IV-Substrates

Glucagon, GIP and the GIP analogs were synthesized with an automated synthesizer SYMPHONY (RAININ) using a modified Fmoc-protocol. Cycles were modified by using double couplings from the 15^{th} amino acid from the C-terminus of the peptide with five-fold excess of Fmoc-amino acids and coupling reagent. The peptide couplings were performed by TBTU/NMM-activation using a 0.23 mmol substituted NovaSyn TGR-resin or the corresponding preloaded Wang-resin at 25 μmol scale. The cleavage from the resin was carried out by a cleavage-cocktail consisting of 94.5% TFA, 2.5% water, 2.5% EDT and 1% TIS.

Analytical and preparative HPLC were performed by using different gradients on the LiChrograph HPLC system of Merck-Hitachi. The gradients were made up from two solvents: (A) 0.1% TFA in H₂O and (B) 0.1% TFA in acetonitrile. Analytical HPLC were performed under the following conditions: solvents were run (1 ml/min) through a 125-4 Nucleosil RP18-column, over a gradient from 5%-50% B over 15 min and then up to 95% B until 20 min, with UV detection (λ=220 nm). Purification of the peptides was carried out by preparative HPLC on either a 250-20 Nucleosil 100 RP8-column or a 250-10 LiChrospher 300 RP18-column (flow rate 6 ml/min, 220 nm) under various conditions depending on peptide chain length. For the identification of the peptide analogues, laser desorption mass spectrometry was employed using the HP G2025 MALDI-TOF system of Hewlett-Packard.

### Example 6

### Computer-Assisted Model for Specificity Examinations of Proline-Specific Proteases

By means of homology modeling approaches a tertiary-structure-models of human DP IV and porcine DP IV have been developed.

The structure of prolyl oligopeptidase (Fülöp, V., et al. (1998) Prolyl Oligopeptidase: An unusual β-propeller domain regulates proteolysis. Cell 94, 161-170) (Brookhaven Protein Data Bank entry: 1 qfm) was used as a target to model the structure of DP IV.

COMPOSER (Blundell, T. L.; Sibanda, B. L.; Sternberg, M. J. E.; Thornton, J. M. Knowledge-based prediction of protein structures and the design of novel molecules. Nature 1987, 326, 347-352; Blundell, T. L.; Carney, D.; Gardner, S.; Hayes, F.; Howlin, B.; Hubbard, T.; Overington, J.; Singh, D. A.; Sibanda, B. L.; Sutcliffe, M. Knowledge-based protein modelling and design. Eur. J. Biochem. 1988, 172, 513-520) a program for homology modeling which is included in the molecular graphics program package SYBYL (TRIPOS Associates Inc., 1699 S. Hanley Road, Suite 303, St. Louis, Mo. 63144) (TRIPOS Associates Inc.) was used to generate the model of DP IV. The amino acid sequences were aligned using the BLOSUM30 matrix (Henikoff, S.; Henikoff, J. G. Amino acid substitution matrices from protein blocks. Proc. Natl. Acad. Sci. USA, (1992), 89, 10915-10919). Afterwards, the modeling procedure consisted of the following steps: structurally conserved regions (SCRs) were identified and a framework of conserved regions was defined as mean positions of structurally equivalent C□-atoms. Structurally variable regions (SVRs, loops) were selected from a program attached database of peptide fragments in order to satisfy end-to-end distances of the SCRs already positioned in the framework. Loops which could not be formed with this procedure were added manually to complete the structure. The conformations of these loops (mainly in the propeller domain) were determined by simulated annealing techniques in heating the temperature to 700 K and subsequently cooling to 100 K by fixing the remaining part of the structure. This procedure was repeated 30 times. All resulting low temperature structures were minimized using the Kollman all-atom force field (Weiner, S. J.; Kollman, P. A.; Case, D. A.; Singh, U. C.; Ghi, C.; Alagona, G.; Profeta, S.; Weiner, P. A new Force Field for molecular mechanical simulation of nucleic acids and proteins, J. Am. Chem. Soc., 1984, 106,765-784). Loop conformations with the lowest energy which fulfill all criteria by analyzing the stereo-chemical quality of the protein structure by means of PROCHECK (Laskowski, R. A. et al. (1993) PROCHECK: a program to check the stereochemical quality of protein structures, J. Appl. Cryst. 26, 283-289) were used.

Small molecule ligands such as substrates of the type Xaa-Pro-p-Nitroanilide were docked with the “automatic” docking program GOLD (C. Bissantz, G. Folkers, D. Rognan; J. Med. Chem. 43, 4759-4767, 2000) to the catalytically active site of DP IV to inspect and analyze the principal correctness of the tertiary structure. Ligands such as GIP or glucagon and longer peptides of the GRF family were docked by application of molecular dynamics simulations. These simulations were started to form a random conformation of these compounds, manual positioned at the outer side of the pore formed by the propeller domain. A low force constant between the protonated N-terminus of the ligands and the side chain of Glu668, which is proposed to be the responsible residue for the recognition of the N-terminus of DP IV was added. Molecular dynamics simulations at 300 K for 100 ps using the Kollman all-atom force field were performed by fixing the backbone atoms of DP IV. All these longer peptides reached the catalytically active site (amino acid position S630), showing that ligands are penetrating through the propeller domain to dock to the active site. The resulting docking structures were optimized and subsequently analyzed to define the so called second binding site of DP IV-substrates.

### Example 7

### Validation of the Computer-Assisted Model of DP IV

Glycosylation Sites

The following residues are assumed to be glycosylated and are therefore placed at the surface of the protein: Asn85, Asn92, Asn150, Asn219, Asn229, Asn281, Asn321, Asn520 and Asn685, which are displayed in FIG. 3. All these amino acid residues are accessible except Asn150 and 321, which are slightly buried but may become accessible by thermal moving of the loop region close to this position.

ADA-Binding Site

Site directed mutagenesis studies proved that the residues L294, V341 and R343 play an important role in ADA binding to DP IV. Therefore, these residues have to be accessible too. These amino acid residues are displayed in FIG. 3. All these residues are situated at the surface of the protein and interact with ADA.

Binding of Small Inhibitors to the Active Site of DP IV

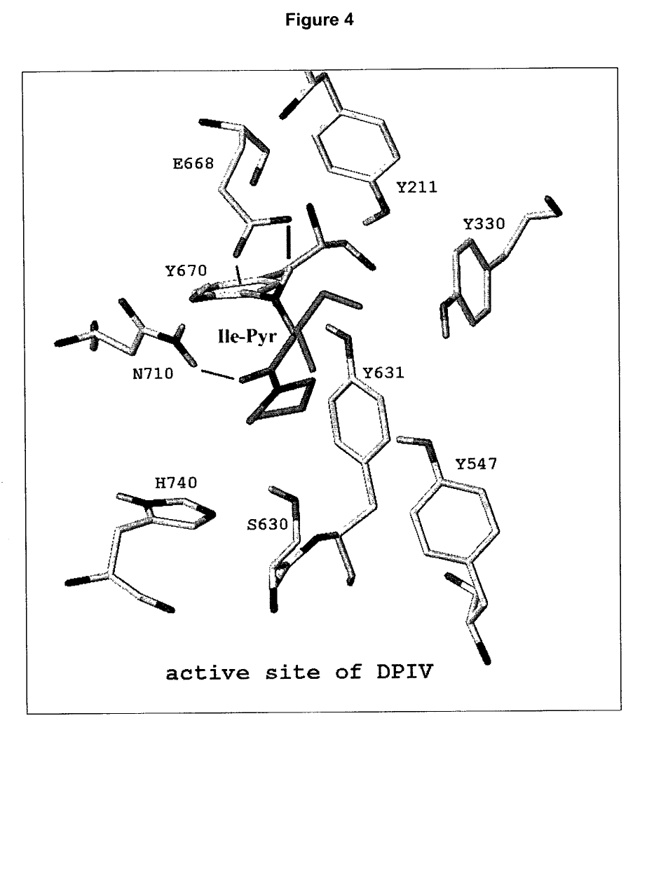

A number of Xaa-Pyrrolidine and Xaa-Proline dipeptides where docked to DP IV and their preferred interaction with the active site was examined (FIG. 4). One of the most important region is the proline recognition site. In POP this site is formed by the two to three amino acid residues. In analogy to POP the proline binding pocked in DP IV is formed also by two aromatic side chains, the two tyrosine residues Y670 and Y631 and by the hydrophobic residue V711.

The S2-binding site in DP IV must be responsible for the recognition of the protonated and positively charged N-terminus of DP IV ligands and preferred interactions of hydrophobic residues such as Val or Ile. The model shows that the side chain of Glu668 is able to form a salt bridge to the N-terminus of ligands. The recognition of the side chains is realized by interactions with the side chains of two other tyrosine residues (Y211 and Y330) and explains the preferred hydrophobic P2-residues of inhibitors.

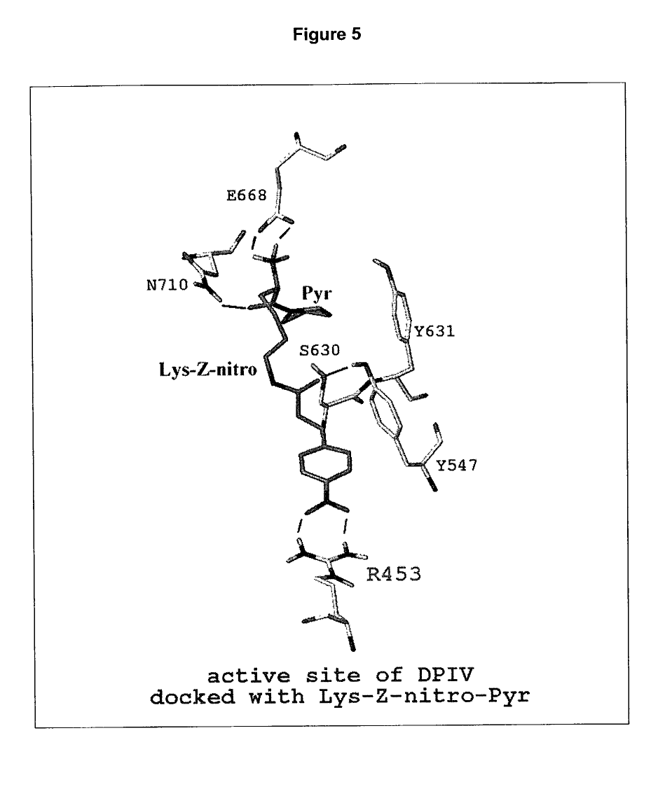

Another DP IV-inhibitor, Lys(Z-nitro)-Pyrrolidine, which carries not a completely hydrophobic P2-side chain, was also docked to DP IV. The result is represented in FIG. 5. In the most stable docking arrangement a scorpion like conformation of the Lys-Z-nitro group can be observed, which finally leads to the formation of a strong hydrogen bond to R453. This additional interaction in comparison to usual dipeptide related ligands explains the high affinity and action of this compound.

Substrate Interactions and Aspects of the Catalytic Mechanism

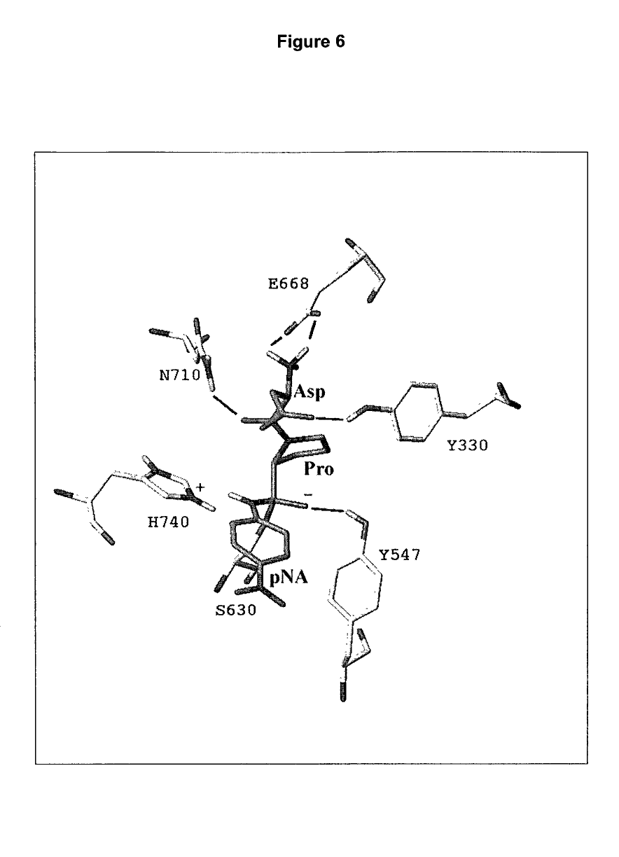

The mode of interaction of substrates to DP IV is shown in FIG. 6. First, the substrates dock exactly in the following conformation: A hydrogen bond is formed between the N—H group and the carbonyl group (torsional angle □2˜80°) of the first amino acid residue (C7-conformation) and the N-terminal amino group is turned out of a □1 torsion of 180° to about 120°. The scissile bond or better plane of the peptide bond to be cleaved is in a perpendicular orientation to the active serine side chain (S630) and allows the reactive attack of the serine to the peptide bond.

Of main importance is the side chain of Y547. The phenolic hydroxyl group forms a hydrogen bond to the carbonyl group of the scissile peptide bond. This interaction plays a very important role in the stabilisation of the tetrahedral intermediate and therefore in the catalytic mechanism in particular in the acylation step. Another interesting finding by Heins et al. (heins et al., Biochim. Biophys. Acta, 1988, 954(2),161-169) was the fact that in the case of proline (in P1) substrates usually the deacylation is the rate limiting step except, when in P2-position an Asp is introduced. A possible docking arrangement of such a substrate is displayed in FIG. 6. The aspartate side chain forms a hydrogen bond to the phenolic OH-group of Y547. This strong interaction prevents the cleaved dipeptide to move out of the binding site and thus shifts the thermodynamic equilibrium and the activation barrier somewhat to the tetrahedral intermediate site and consequently the acylation rate is considerably reduced and becomes rate limiting.

Docking Behavior of Ligands with Biological Importance

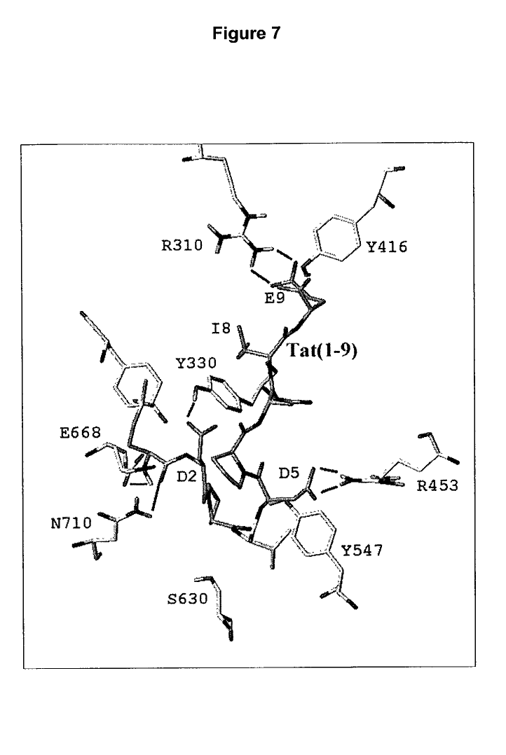

It has been demonstrated that the N-terminal nonapeptide of the HIV-tat protein shows inhibitory effects to DP IV. Docking studies of this compound were done with the complete DP IV model as described above. The resulting most stable binding arrangement is shown in FIG. 7.

There are some important interactions. Similar to the already discussed interaction of the substrate Asp-Pro-PNA D2 of Tat forms a hydrogen bond with Y330 and furthermore as seen for Lys-Z-nitro-Pyrrolidine, D5 forms a salt bridge with R453. Further considerable hydrophobic interactions occur between I 8 and Y330 and another salt bridge is observed between the C-terminal E9 and R310 of DP IV.

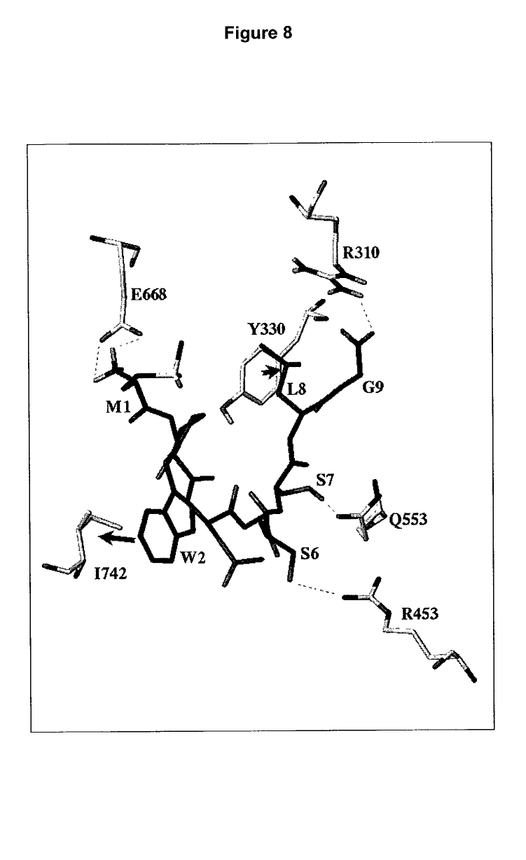

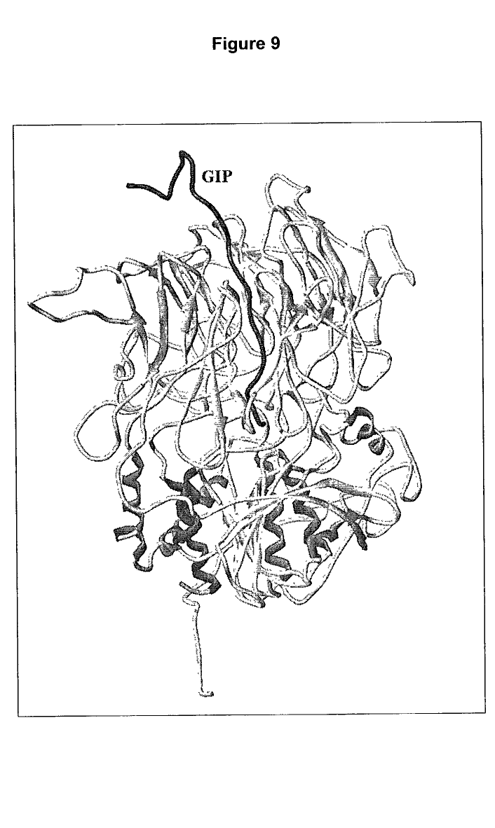

Another similar peptide that was used for docking studies is the N-terminal nonapeptide of the tromboxane receptor (FIG. 8). Similar interactions as seen for HIV-tat were detected. Additionally important is the hydrophobic interaction between W2 and I742.

### Example 8

### Docking of GIP; VIP and Glucagons to DP IV

Several oligopeptides such as GIP, VIP, glucagon and others are hydrolysed by DP IV and therefore it is clear, that these substrates are docking to DP IV and reaching the active site. Extensive docking investigations by means of molecular dynamics simulations were done using the old model. From these studies the structure of a hexapeptide, TFTSDY, was derived and its ability to protect oligopeptide substrates from their interaction with a secondary binding site.

Results

The binding and hydrolysis of small dipeptide substrates were only slightly influenced when DP IV was preincubated with the hexapeptide TFTSDY, but the affinity of oligopeptides such as GIP, VIP, glucagon and others was considerably reduced. These experiments clearly prove the existence of a secondary binding site.

How these rather long peptides reach the active site of DP IV without essential steric hindrance was investigated. GIP was placed at the top of the propeller domain with the N-terminus pointing to the direction middle to DP IV. A small constraint (additional force constant) was placed between the N-terminal nitrogen atom of GIP and a carboxyl oxygen atom of E668. Then a molecular dynamics simulation over 50.000 fs at 300 K was started with fixed backbone atoms of DP IV in the gas phase. Surprisingly it was shown that GIP moved in the pore rapidly without any considerable steric hindrance and was indeed able to reach the active site. Finally starting from the end structure of this “constrained” dynamic model, dynamic simulations with GIP already situated inside DP IV were repeated. The final optimized docking arrangement is shown in FIGS. 9 to 12 and the most important interactions are summarized in Tables 6 to 8.

TABLE 6
Most important interactions of GIP with DP IV

| GIP | DP IV | typ of interaction |
|---|---|---|
| NT | E668 | salt bridge |
| Y1-CO | N710 | H-bond |
| S2-OH | Y631 | H-bond |
| S2-CO | Y547 | H-bond (Catalysis!) |
| E3 | R560 | salt bridge |
| I7 | Y330 | hydrophob |
| D9 | R310 | salt bridge |
| Y10 | W154 | hydrophob |
| I12 | W157 | hydrophob |
| D15 | K463 (R318) | salt bridge |
| K16 | E464 (E91) | salt bridge |

TABLE 7
Most important interactions of VIP with DP IV

| VIP | DP IV | typ of interaction |
|---|---|---|
| NT | E668 | salt bridge |
| H1-CO | N710 | H-bond |
| H1-side chain | I742 | hydrophob |
| S2-OH | Y631 | H-bond |
| S2-CO | Y547 | H-bond (catalysis!) |
| D3 | R560 | salt bridge |
| D8 | R310 | salt bridge |
| Y10 | W154 | hydrophob |
| Y10-CO | S460 | H-bond |
| K15 | E464 | salt bridge |

TABLE 8
Most important interactions of Glucagon with DP IV

| Glucagon | DP IV | typ of interaction |
|---|---|---|
| NT | E668 | salt bridge |
| H1-CO | N710 | H-bond |
| H1-side chain | I742 | hydrophob |
| A2-CO | Y547 | H-bond |
| Q3 | R560 | H-bond |
| T5 | T152 | H-bond |
| T7 | Y416 | H-bond |
| S8 | Y330 | H-bond |
| D9-CO | Y416 | H-bond |
| D9 | R310 | salt bridge |
| Y10 | W154 | hydrophob |
| Y13 | L90 | hydrophob |
| D15 | R318 | salt bridge |
| R17 | E91 | salt bridge |

TABLE 9
Most important interactions of the hexapeptide T(5)-F-T-S-D-Y with
DP IV

| hexapeptide | DP IV | type of interaction |
|---|---|---|
| T5 | T152 | H-bond |
| T7 | Q153(CO-backbone) | H-bond |
| S8 | S552 | H-bond |
| D9 | R310 | salt bridge |
| Y10 | W154 | hydrophobic |
| Y10(OH) | T152(OH) | H-bond |
| Y10(OH) | T152(CO-backbone) | H-bond |
| Y10(CT) | S460(OH) | H-bond |

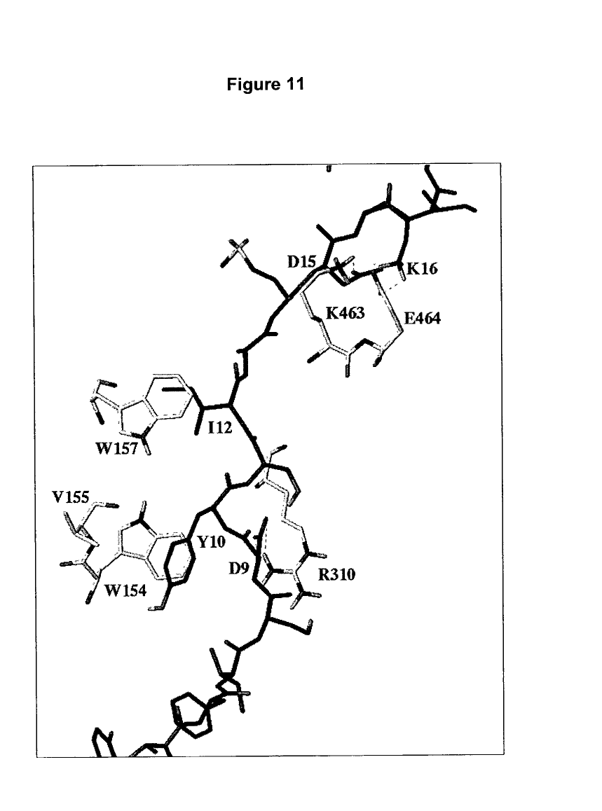

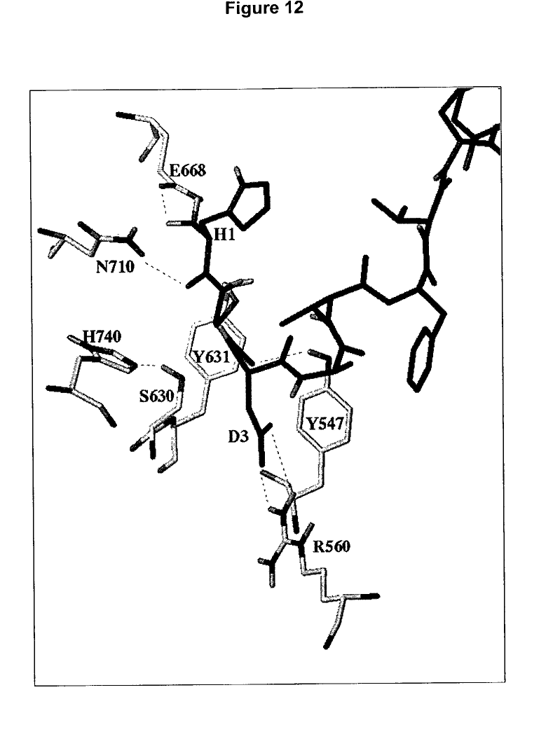

As can be seen in FIGS. 9 and 11, GIP is able to reach the active site of DP IV, but the C-terminal tail is still at the surface of the propeller domain.

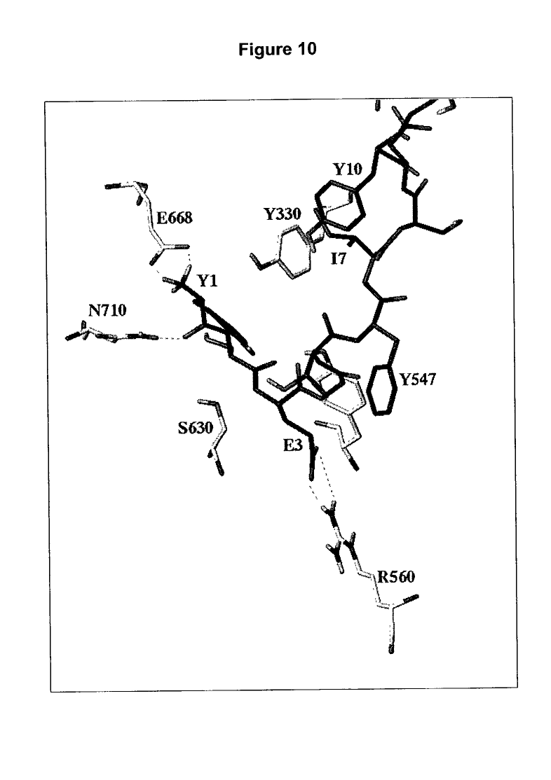

The scissile peptide bond after Ser2 is exactly in an orientation required for optimal hydrolysis (FIG. 10). A number of important interactions which explain the affinity of GIP to DP IV were detected. These attractive interactions are summarized in Table 6. Interestingly a number of interactions were observed, which were already discussed for other ligands (see above).

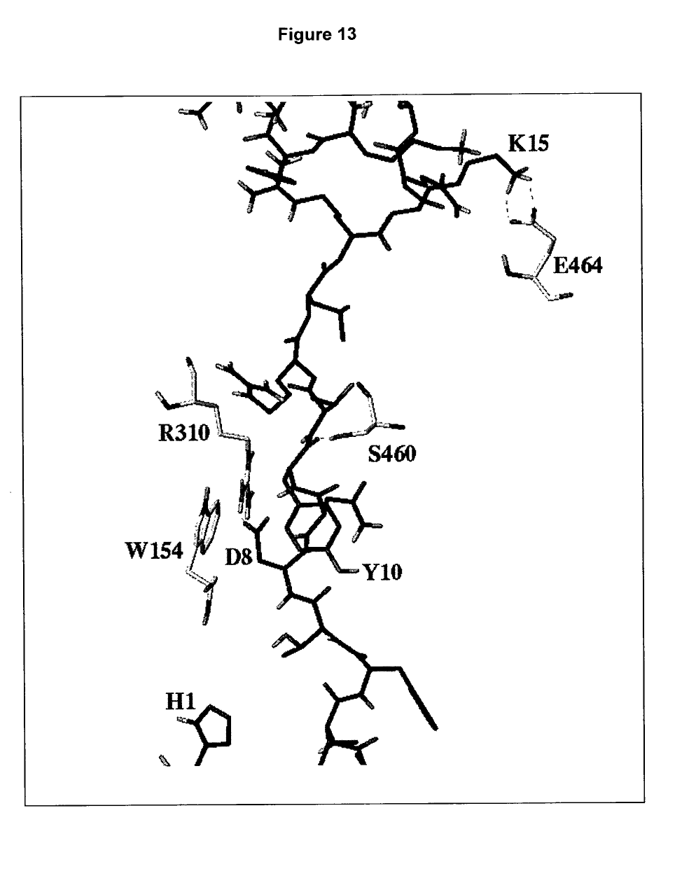

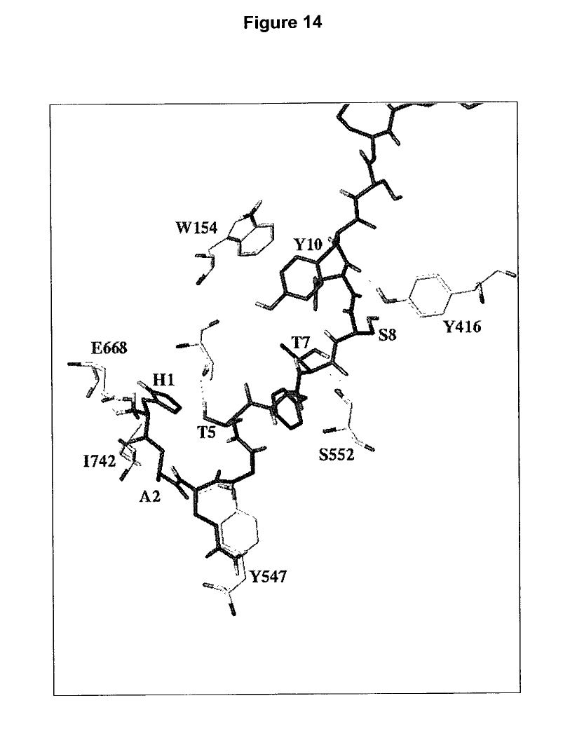

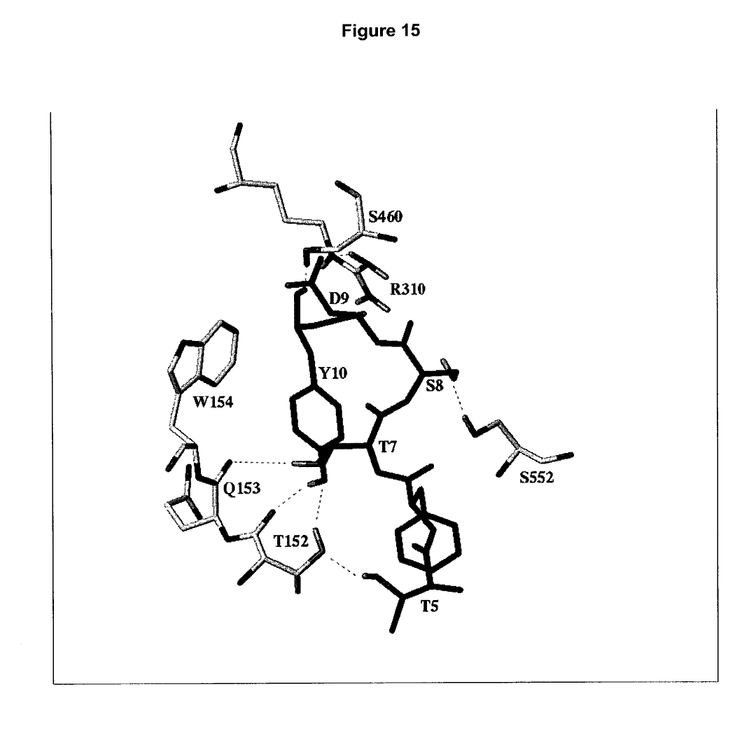

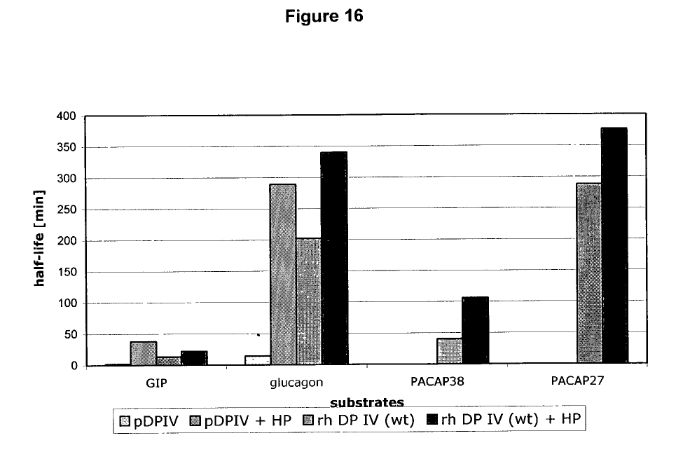

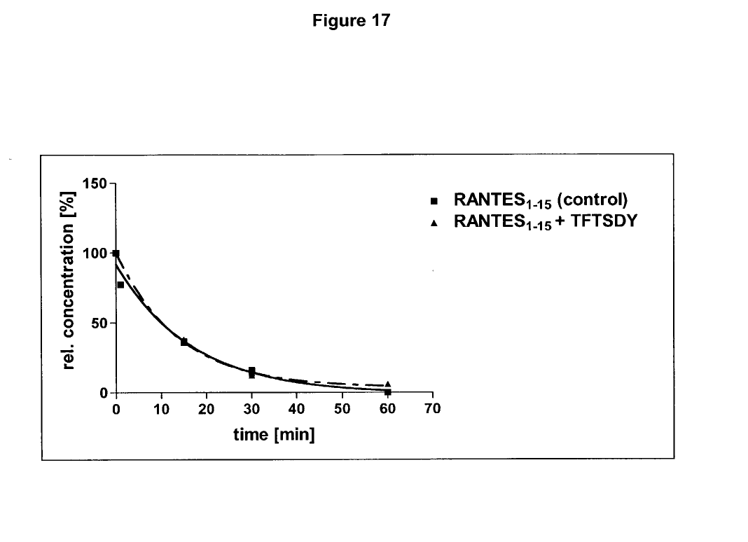

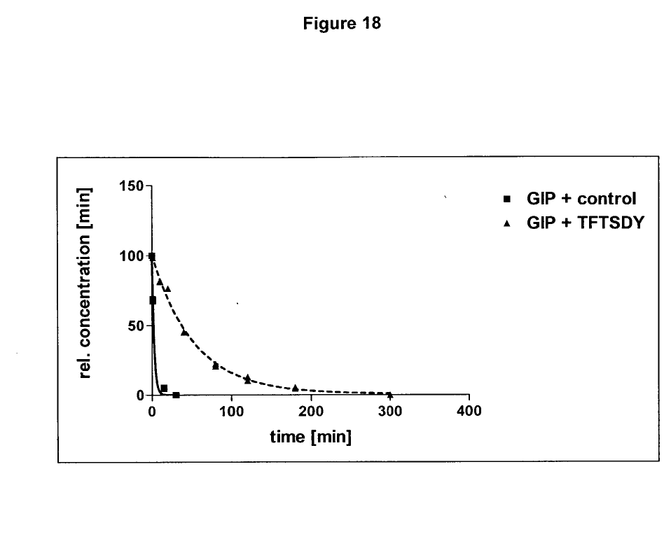

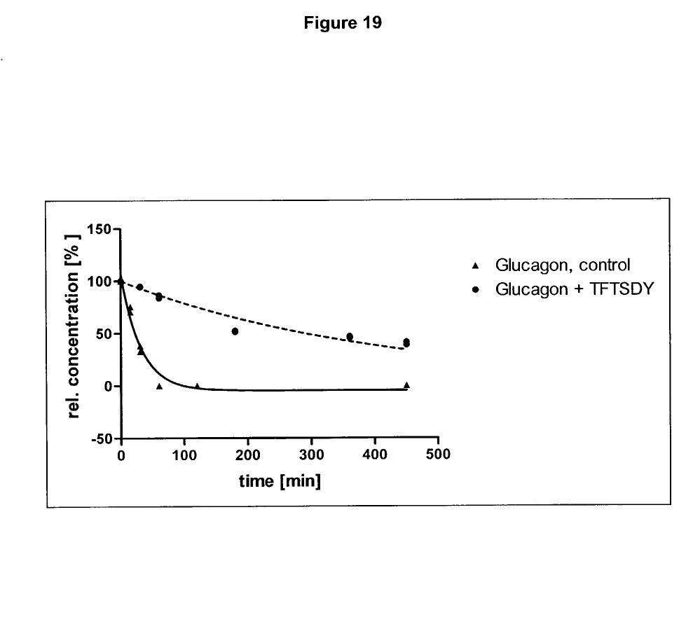

Based on these results analogous docking studies were performed with VIP, glucagon and the hexapeptide TFTSDY (FIGS. 12 to 15) The results are summarized by listing the most attractive interactions in Tables 7 to 9.

These results prove that the oligopeptide ligands penetrate through the propeller domain to dock to the active site. Furthermore, some highly attractive interactions between the oligopeptide ligands and DP IV were shown, which explain the affinity of the calculated compounds and which were used to predict the structure of non-peptidic ligands for the secondary binding site of DP IV. Some preliminary structures of such non-peptidic ligands are provided in the description above.

Moreover, the results of these studies confirm the proposed docking of Lys-Z-nitro-Pyrrolidine, e.g. the interaction of the nitro-group with AR560. Wher the oligopeptide ligands have an Asp in third or fourth position in their amino acid sequence, a salt bridge with R560 is formed. By docking arrangement of the hexapetide TFTSDY (FIG. 15), it was proven that this hexapeptide indeed prevents binding of oligopeptide ligands to the active site.

What is claimed is:

1. Compounds capable of binding to a secondary binding site of dipeptidyl peptidase IV or dipeptidyl peptidase IV-like enzymes and altering the selectivity and/or activity of such enzymes toward their natural substrates.

2. Compounds of claim 1, wherein the secondary binding site comprises but are not restricted to the amino acid residues L90, E91, T152, W154, W157, R310, Y330, R318, Y416, S460, K463, E464 and R560.

3. Compounds according to claim 1, which are selected from the hexapeptides TFTSDY and TFTDDY.

4. Compounds according to claim 1, which are selected from the group of compounds of formulas a) to d):

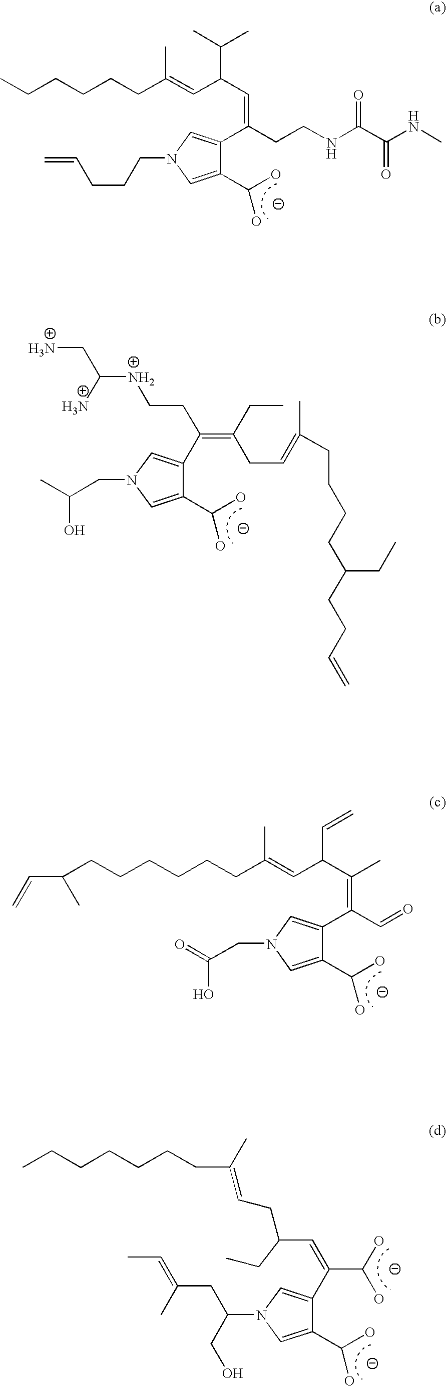

5. A method for modulating the selectivity and/or activity of dipeptidyl peptidase IV or dipeptidyl peptidase IV-like enzymes in a mammal comprising administering to said mammal a therapeutically effective amount of at least one selectivity and/or activity modifying compound capable of binding to a secondary binding site of dipeptidyl peptidase IV or dipeptidyl peptidase IV-like enzymes.

6. A method for modulating the interaction of dipeptidyl peptidase IV or dipeptidyl peptidase IV-like enzymes with binding proteins in a mammal comprising administering a therapeutically effective amount of at least one selectivity and/or activity modifying compound capable of binding to a secondary binding site of dipeptidyl peptidase IV or dipeptidyl peptidase IV-like enzymes.

7. The method according to claim 5, wherein the selectivity and/or activity modifying compound is selected from the hexapeptides TFTSDY and TFTDDY.

8. The method according to claim 6, wherein the selectivity and/or activity modifying compound is selected from the hexapeptides TFTSDY and TFTDDY.

9. The method according to claim 5, wherein the selectivity and/or activity modifying compound is selected from the group of compounds of formulas a) to d):

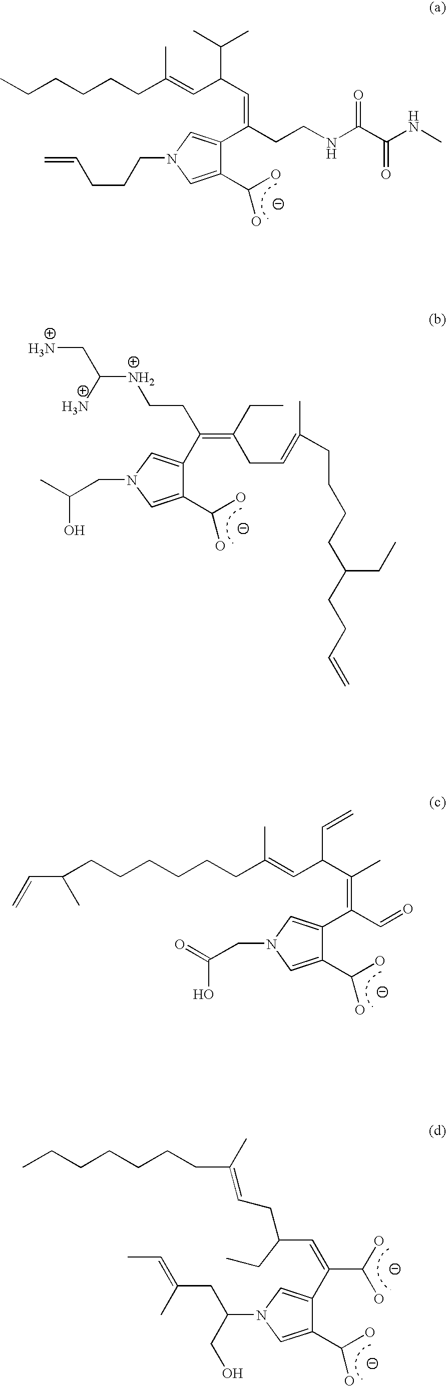

10. The method according to claim 6, wherein the selectivity and/or activity modifying compound is selected from the group of compounds of formulas a) to d):

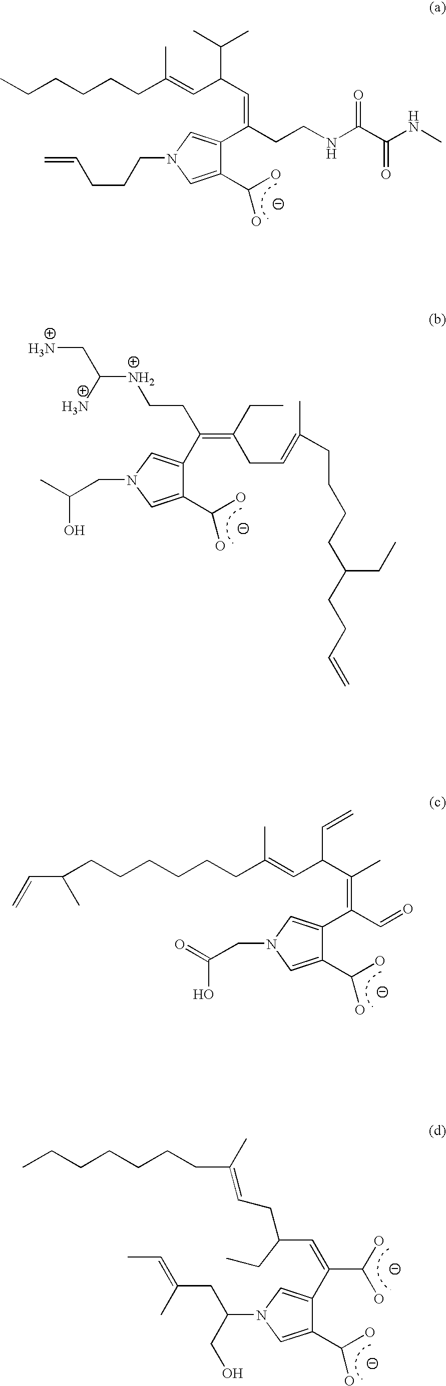

11. The method according to claim 5, wherein the selectivity and/or activity modifying compound binds to both, the active site and the secondary binding site of dipeptidyl peptidase IV or dipeptidyl peptidase IV-like enzymes.

12. The method according to claim 6, wherein the selectivity and/or modifying compound binds to both, the active site and the secondary binding site of dipeptidyl peptidase IV or dipeptidyl peptidase IV-like enzymes.

13. A composition containing a selectivity and/or activity modifying compound according to claim 1 and a DP IV-inhibitor.

14. A composition containing a selectivity and/or activity modifying compound according to claim 2 and a DP IV-inhibitor.

15. A composition containing a selectivity and/or activity modifying compound according to claim 3 and a DP IV-inhibitor.

16. A composition containing a selectivity and/or activity modifying compound according to claim 4 and a DP IV-inhibitor.

17. The method according to claim 5, wherein the selectivity and/or activity modifying compound is administered in combination with a DP IV-inhibitor.

18. The method according to claim 6, wherein the selectivity and/or activity modifying compound is administered in combination with a DP IV-inhibitor.

19. The method according to claim 7, wherein the selectivity and/or activity modifying compound is administered in combination with a DP IV-inhibitor.

20. The method according to claim 8, wherein the selectivity and/or activity modifying compound is administered in combination with a DP IV-inhibitor.

21. The method according to claim 9 wherein said mammal is a human and wherein said modulation results in the treatment of metabolic diseases of humans.

22. The method according to claim 10 wherein said mammal is a human and wherein said modulation results in the treatment of metabolic diseases of humans.

23. The method according to claim 17 for the treatment of impaired glucose tolerance, glucosuria, hyperlipidaemia, metabolic acidosis, diabetes mellitus, diabetic neuropathy and nephropathy and of sequelae caused by diabetes mellitus, neurodegenerative diseases and disturbance of signal action at the cells of the islets of Langerhans and insulin sensitivity in the peripheral tissue in the postprandial phase of mammals.

24. The method according to claim 18 for the treatment of impaired glucose tolerance, glucosuria, hyperlipidaemia, metabolic acidosis, diabetes mellitus, diabetic neuropathy and nephropathy and of sequelae caused by diabetes mellitus, neurodegenerative diseases and disturbance of signal action at the cells of the islets of Langerhans and insulin sensitivity in the peripheral tissue in the postprandial phase of mammals.

25. The method according to claim 17 for the treatment of metabolism-related hypertension and cardiovascular sequelae caused by hypertension in mammals.

26. The method according to claim 18 for the treatment of metabolism-related hypertension and cardiovascular sequelae caused by hypertension in mammals.

27. The method according to claim 17 for the prophylaxis or treatment of skin diseases and diseases of the mucosae, autoimmune diseases and inflammatory conditions.

28. The method according to claim 18 for the prophylaxis or treatment of skin diseases and diseases of the mucosae, autoimmune diseases and inflammatory conditions.

29. The method according to claim 17 for the treatment of psychosomatic, neuropsychiatric and depressive illnesses, such as anxiety, depression, sleep disorders, chronic fatigue, schizophrenia, epilepsy, nutritional disorders, spasm and chronic pain.

30. The method according to claim 18 for the treatment of psychosomatic, neuropsychiatric and depressive illnesses, such as anxiety, depression, sleep disorders, chronic fatigue, schizophrenia, epilepsy, nutritional disorders, spasm and chronic pain.

31. The method according to claim 17 for the chronic treatment of chronic metabolic diseases in humans.

32. The method according to claim 18 for the chronic treatment of chronic metabolic diseases in humans.

33. The method according to claim 17 for the chronic treatment of chronically impaired glucose tolerance, chronic glucosuria, chronic hyperlipidaemia, chronic metabolic acidosis, chronic diabetes mellitus, chronic diabetic neuropathy and nephropathy and of chronic sequelae caused by diabetes mellitus, chronic neurodegenerative diseases and chronic disturbance of signal action at the cells of the islets of Langerhans and chronic insulin sensitivity in the peripheral tissue in the postprandial phase of mammals.

34. The method according to claim 18 for the chronic treatment of chronically impaired glucose tolerance, chronic glucosuria, chronic hyperlipidaemia, chronic metabolic acidosis, chronic diabetes mellitus, chronic diabetic neuropathy and nephropathy and of chronic sequelae caused by diabetes mellitus, chronic neurodegenerative diseases and chronic disturbance of signal action at the cells of the islets of Langerhans and chronic insulin sensitivity in the peripheral tissue in the postprandial phase of mammals.

35. The method according to claim 17 for the chronic treatment of metabolism-related hypertension and of chronic cardiovascular sequelae caused by chronic hypertension in mammals.

36. The method according to claim 18 for the chronic treatment of metabolism-related hypertension and of chronic cardiovascular sequelae caused by chronic hypertension in mammals.

37. The method according to claim 17 for the chronic treatment of chronic psychosomatic, chronic neuropsychiatric and depressive illnesses, such as chronic anxiety, chronic depression, chronic sleep disorders, chronic fatigue, chronic schizophrenia, chronic epilepsy, chronic nutritional disorders, spasm and chronic pain.

38. The method according to claim 18 for the chronic treatment of chronic psychosomatic, chronic neuropsychiatric and depressive illnesses, such as chronic anxiety, chronic depression, chronic sleep disorders, chronic fatigue, chronic schizophrenia, chronic epilepsy, chronic nutritional disorders, spasm and chronic pain.

39. A method for screening compounds capable of binding to a secondary binding site of DP IV or DP IV-like enzymes comprising the following steps:
a) Contacting said compounds with DP IV or a DP IV-like enzyme, preferably under conditions which would permit binding therebetween;
b) Adding a substrate of DP IV or DP IV-like enzymes to said DP IV or DP IV-like enzyme;
c) Monitoring the biodegradation of the substrate or optionally measuring the residual DP IV or DP IV-like enzyme activity; and
d) Correlating changes in the biodegradation and/or enzyme activity with the binding of said compounds to DP IV or DP IV-like enzymes to identify an activity modifying agent.

40. A method for detecting the presence of secondary binding site(s) of DP IV and DP IV-like enzymes comprising the following steps:
a) providing two or more substrates, having an amino acid sequence, binding to DP IV or DP IV-like enzymes and aligning the amino acid sequences of said substrates;
b) identifying at least one consensus sequence amongst said substrate amino acid sequences;
c) synthesizing a peptide having said consensus sequence;
d) contacting said synthesized peptide with DP IV or a DP IV-like enzyme;
e) adding a substrate of DP IV or DP IV-like enzymes to the DP IV or DP IV-like enzyme;
f) monitoring the biodegradation of the substrate or optionally measuring the residual DP IV or DP IV-like enzyme activity; and
g) correlating changes in said biodegradation or enzyme activity with the presence of a second binding site capable of modulating the substrate specificity of DP IV or DP IV-like enzymes.
